(19) United States
(12) Patent Application Publication
(10) Pub. No.: US 2011/0300078 A1
(43) Pub. Date: Dec. 8, 2011

(54) ISOLATION OF MICROBUBBLES OF SELECTED SIZE RANGE FROM POLYDISPERSE MICROBUBBLES

(75) Inventors: Mark A. Borden, New York, NY (US); Jameel Adebayo Feshitan, New York, NY (US); Cherry Chen, New York, NY (US); Shashank Ramesh Sirsi, New York, NY (US); James Jing Kwan, Hartsdale, NY (US)

(73) Assignee: THE TRUSTEES OF CLUMBIA UNIVERSITY IN THE CITY OF NEW YORK, Broadway, NY (US)

(21) Appl. No.: 13/044,224

(22) Filed: Mar. 9, 2011

Related U.S. Application Data
(63) Continuation-in-part of application No. PCT/US09/56513, filed on Sep. 10, 2009.
(60) Provisional application No. 61/095,933, filed on Sep. 10, 2008.

(51) Int. Cl. A61K 49/18; A61K 9/00; B01D 43/00

(52) U.S. Cl. 424/9.52; 494/37; 424/400

(57) ABSTRACT

In one aspect of the disclosed subject matter, a method for isolating target microbubbles having a predetermined size range from polydisperse microbubbles is disclosed. The method includes applying a first centrifugal field having a first field strength to a suspension comprising the polydisperse microbubbles for a first duration of time, thereby forming a first infranatant comprising at least a portion of target microbubbles and a first supernatant cake comprising microbubbles having a greater size than the target microbubbles; removing the first supernatant cake; applying a second centrifugal field having a second field strength to the first infranatant for a second duration of time, the second field strength being greater than the first field strength, thereby forming a second supernatant cake comprising at least a portion of the target microbubbles and second infranatant comprising microbubbles having a smaller size than the target microbubbles; and isolating the second supernatant cake. In another aspect of the disclosed subject matter, a method for performing high frequency ultrasonic imaging using isolated microbubbles is provided.

### RELATED APPLICATIONS

The present application is a continuation-in-part application of International Application PCT/US09/056,513, filed Sep. 10, 2009, which claims priority to U.S. Provisional Application Ser. No. 61/095,933, filed on Sep. 10, 2008, the disclosure of each of which is incorporated by reference herein in its entirety.

### STATEMENT REGARDING FEDERALLY SPONSORED RESEARCH

This invention was made with government support under grant numbers R01-EB009066 and R21-CA139173, both awarded by the National Institutes of Health. The government has certain rights in this invention.

### BACKGROUND

Microbubbles are being employed for several biomedical applications, including contrast enhanced ultrasound, drug and gene delivery, and metabolic gas delivery. Microbubbles can react strongly to ultrasonic pressure waves by virtue of their compressible gas cores, which resonate at the MHz-frequencies used by current clinical scanners. Oscillation of the gas core allows re-radiation (backscatter) of ultrasound energy to the transducer at harmonic frequencies and nonlinear modes, thus providing exquisite sensitivity in detection with current contrast-enhanced pulse sequences and signal processing algorithms. Additionally, microbubbles can cavitate stably or inertially to facilitate drug release and extravascular delivery within the transducer focus.

Current commercially available microbubble formulations can be polydisperse in size. In some cases, the size distribution is broad over a range of submicron to tens of μm in diameter. This can be problematic because microbubble behavior depends on size. For example, increasing the microbubble diameter from 1 to 5 μm will change the resonance frequency of an unencapsulated microbubble from 4.7 to 0.72 MHz. Microbubble size can also impact biodistribution and pharmacodynamics after intravenous injection, bioeffects during ultrasound insonification, gas release profile, and other related behaviors. Therefore, microbubbles of a specific size with low polydispersity are desired for advanced biomedical applications.

Techniques for producing or isolating monodisperse microbubbles have been in development. For example, microfluidic technologies have been used to engineer monodisperse microbubble suspensions. These techniques include flow focusing, T-junctions and electrohydrodynamic atomization. While these techniques can provide for low polydispersity, they can be slow at generating microbubbles. Using flow focusing, for example, can require several hours to produce microbubbles at sufficient numbers for even a single small-animal trial (˜0.1 mL×10⁹ mL⁻¹). Additionally, dust particles can plug micro-channels, thus requiring fabrication and calibration of a new device.

Mechanical agitation is one method to create encapsulated microbubbles for biomedical applications. It is an emulsification procedure in which a hydrophobic phase (i.e., gas) is dispersed within an aqueous surfactant solution by disruption of the interface. Acoustic emulsification (sonication), for example, can generate large quantities of microbubbles (e.g., 100 mL×10¹⁰ mL⁻¹) rapidly and reproducibly within just a few seconds. Shaking a serum vial with a device similar to a dental amalgamator produces a sufficient dose of microbubbles (2 mL×10¹⁰ mL⁻¹) for a single patient study, at the bedside in less than a minute. However, the size distributions of the microbubbles generated by mechanical agitation are highly polydisperse.

Accordingly, there is a need for an efficient method for isolating selected size fractions of microbubbles of interest with sufficient yield from polydisperse microbubbles for biomedical applications.

### SUMMARY

The disclosed subject matter provides techniques for isolating target microbubbles having a predetermined size range from polydisperse microbubbles, as well as for performing ultrasonic imaging using the size-isolated microbubbles.

In one aspect of the disclosed subject matter, a method for isolating target microbubbles having a predetermined size range from polydisperse microbubbles is provided. The method includes: applying a first centrifugal field having a first field strength to a suspension comprising the polydisperse microbubbles for a first duration of time, thereby forming a first infranatant comprising at least a portion of target microbubbles and a first supernatant cake comprising microbubbles having a greater size than the target microbubbles; removing the first supernatant cake; applying a second centrifugal field having a second field strength to the first infranatant for a second duration of time, the second field strength being greater than the first field strength, thereby forming a second supernatant cake comprising at least a portion of the target microbubbles and second infranatant comprising microbubbles having a smaller size than the target microbubbles; and isolating the second supernatant cake.

In some embodiments of the above method, the applied first centrifugal field and the first duration of time can be sufficient to cause substantially all the microbubbles in the polydisperse microbubbles having a greater size than the target microbubbles to form the first supernatant cake. In these embodiments, the applied second centrifugal field and the second duration of time can be sufficient to cause substantially all the target microbubbles to form the second supernatant cake. The isolated second supernatant cake can be further redispersed into a new dispersion, which can be subjected to a third centrifugal field having a third field strength for a third duration of time, thereby forming a third supernatant cake comprising at least a portion of the target microbubbles, and then the third supernatant cake is isolated.

In some embodiments of the method, the polydisperse microbubbles include microbubbles having sizes from smaller than about 1 μm to larger than about 10 μm. In certain embodiments, the target microbubbles have a size range of about 4 μm to about 5 μm. In other embodiments, the target microbubbles have a size range of about 1 μm to about 2 μm.

In some embodiments of the method, the polydisperse microbubbles are coated at least in part with lipids. In other embodiments, the polydisperse microbubbles are coated at least in part with polymeric surfactants.

In some embodiments of the method, the core of the polydisperse microbubbles comprises perfluorobutane.

In some embodiments of the method, the polydisperse microbubbles are obtained by sonication. In certain embodiments, the polydisperse microbubbles have a multimodal size distribution.

In some embodiments of the method, the microbubble suspension can be placed in a syringe when being subjected to a centrifugal field. The syringe has a longitudinal axis, a length, a cap, and a drainage portion. The drainage portion of the syringe is positioned further away from the central axis of the centrifugal field relative to the cap.

In some embodiments of the disclosed subject matter, a method for isolating target microbubbles having a predetermined size range from polydisperse microbubbles is provided. The method includes: applying a first centrifugal field having a first field strength to a suspension comprising the polydisperse microbubbles for a first duration of time, thereby forming a first infranatant comprising at least a portion of target microbubbles and a first supernatant cake comprising microbubbles having a greater size than the target microbubbles; removing the first supernatant cake; applying a second centrifugal field having a second field strength to the first infranatant for a second duration of time, the second field strength being greater than the first field strength, thereby forming a second infranatant comprising at least a portion of target microbubbles and a second supernatant cake comprising microbubbles having a greater size than the target microbubbles; removing the second supernatant cake; applying a third centrifugal field having a third field strength to the second infranatant for a third duration of time, the third field strength being greater than the second field strength, thereby forming a third supernatant cake comprising at least a portion of the target microbubbles and third infranatant comprising microbubbles having a smaller size than the target microbubbles; and isolating the third supernatant cake.

In some embodiments of the above method, the applied second centrifugal field and the second duration of time can be sufficient to cause substantially all the microbubbles in the first infranatant having a greater size than the target microbubbles to form the second supernatant cake. In these embodiments, the applied third centrifugal field and the third duration of time can be sufficient to cause substantially all the target microbubbles to form the third supernatant cake. The isolated third supernatant cake can be further redispersed into a new dispersion, which can be subjected to a fourth centrifugal field having a fourth field strength for a fourth duration of time, thereby forming a fourth supernatant cake comprising at least a portion of the target microbubbles, and then the fourth supernatant cake can be isolated.

In some embodiments of the above methods, the total volume fraction of the microbubbles in the polydisperse microbubbles suspension and each of the subsequently formed supernatant can be equal to or below about 20%, and at least applying one of the centrifugal fields comprises first determining the centrifugal field strength to be applied using a Stoke's equation. The Stoke's equation correlates the rise velocity of a microbubble in a suspension relative to the bulk fluid under creeping flow conditions with the size of the microbubble, the centrifugal field strength to be applied, and the effective viscosity of the suspension. The determination of the respective centrifugal field strengths can be based at least on the duration of time during which the respective centrifugal field is to be applied.

In another aspect of the disclosed subject matter, monodisperse microbubbles having a predetermined size range prepared by the above described methods are provided.

In yet another aspect of the disclosed subject matter, a method of performing high frequency ultrasonic imaging is provided. The method includes: administering monodisperse microbubbles having an number-averaged size of between about 1 to 10 μm to an animal; and performing ultrasonic imaging on the animal at a fundamental frequency of at least about 30 MHz. The monodisperse microbubbles for performing the high frequency ultrasonic imaging can have a size range of about 1 μm to about 2 μm, about 4 μm to about 5 μm, or about 6 μm to about 8 μm.

### BRIEF DESCRIPTION OF THE DRAWINGS

For a more complete understanding of example embodiments of the described subject matter and its advantages, reference is now made to the following description, taken in conjunction with the accompanying drawings, in which:

FIG. 1 depicts a block diagram illustrating a method for isolating target microbubbles according to some embodiments of the disclosed subject matter.

FIG. 2 depicts a schematic diagram of differential centrifugation for the size isolation of microbubbles.

FIG. 3 depicts size distributions of freshly sonicated microbubbles according to some embodiments of the disclosed subject matter.

FIG. 4 depicts microscopy images of initial polydisperse and size-isolated microbubbles according to some embodiments of the disclosed subject matter.

FIG. 5 depicts plots illustrating size distribution of initial polydisperse and size-isolated microbubbles determined by flow cytometry according to some embodiments of the disclosed subject matter.

FIG. 6 depicts plots illustrating size distributions of initial polydisperse and size-isolated microbubbles determined by light scattering and electrozone sensing, respectively, according to some embodiments of the disclosed subject matter.

FIG. 7 depicts fluorescence intensity and light scattering profiles for microbubble suspensions after size isolation as determined by flow cytometry according to some embodiments of the disclosed subject matter.

FIG. 8 depicts the stability of size-isolated microbubbles according to some embodiments of the disclosed subject matter.

FIG. 9 depicts of size distributions of microbubble suspensions according to some embodiments of the disclosed subject matter.

FIG. 10 depicts size distributions of size-selected microbubbles according to some embodiments of the disclosed subject matter.

FIG. 11 illustrates contrast enhancement in ultrasound imaging as a result of using size-selected microbubbles according to some embodiments of the disclosed subject matter.

FIG. 12 illustrates attenuation of ultrasound signal from 1-2 μm microbubbles in an ultrasound imaging experiment according to some embodiments of the disclosed subject matter.

FIG. 13 depicts time-intensity curves of ultrasound signal using polydisperse and size-selected microbubble suspensions according to some embodiments of the disclosed subject matter.

FIG. 14 depicts signal amplitude and half-life of size-selected microbubbles according to some embodiments of the disclosed subject matter.

FIG. 15 depicts signal amplitude and half-life of size-selected microbubbles as a function of total gas volume, according to some embodiments of the disclosed subject matter.

FIG. 16 depicts certain parameters for model fit of time-intensity curves (TIC) of ultrasound imaging using size-selected microbubbles according to some embodiments of the disclosed subject matter.

FIG. 17 depicts extinction cross-section as a function of diameter (in 17A), and the effect of surface tension on extinction cross-section (in 17B), according to some embodiments of the disclosed subject matter.

### DETAILED DESCRIPTION

In one aspect, the disclosed subject matter provides techniques for isolating a selected size fraction of microbubbles (or target microbubbles) from polydisperse microbubbles. First, a dispersion (or suspension) of microbubbles having broad size distribution can be generated or obtained. These initial microbubbles are then subjected to at least two stages of separation. In the first stage, the portion of the microbubbles having a greater size than the target microbubbles are separated from the initial microbubbles and discarded. In the second stage, the remaining microbubbles are further separated into two portions: a first portion containing substantially all the target microbubbles, and the other portion containing mostly microbubbles smaller than the target microbubbles. The first and the second stage can both be divided into several sub-stages of separation to optimize the separation result.

The presently described techniques allow rapid and robust production of a narrowly distributed (or monodisperse) microbubbles of a desired size range from polydisperse microbubbles that can be generated in large quantities by simple mechanical agitation. Both of the generation of initial polydisperse microbubbles and the size-isolation methods as described herein can be conveniently and inexpensively performed at the site where the target microbubbles are intended to be used.

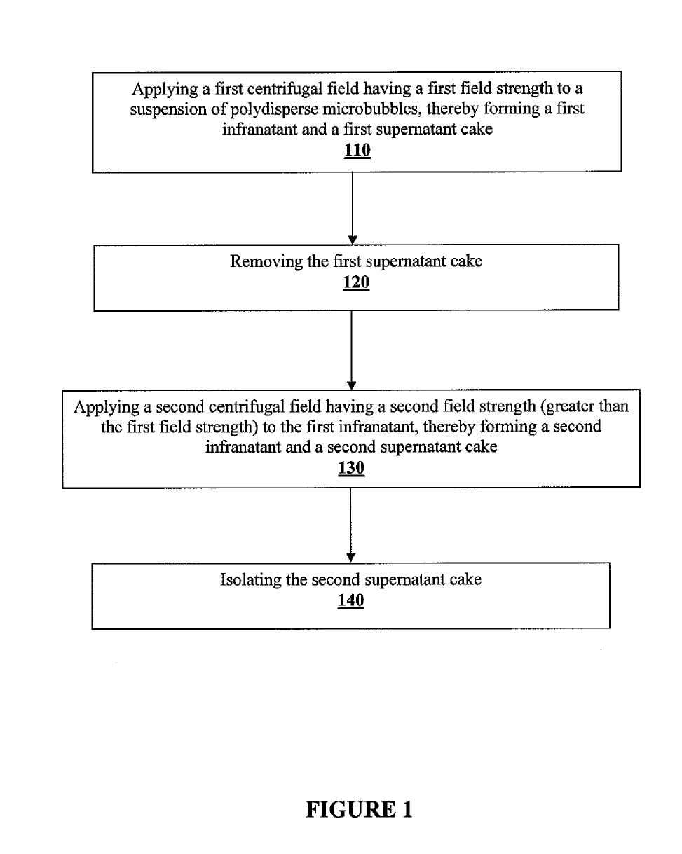

Referring to FIG. 1, a method for isolating target microbubbles having a predetermined size range from polydisperse microbubbles according to some embodiments of the disclosed subject matter will be explained. At 110, a first centrifugal field having a first field strength is applied to a suspension comprising the polydisperse microbubbles for a first duration of time, thereby forming a first infranatant and a first supernatant cake. The first infranatant is a suspension that includes at least a portion of target microbubbles; and the first supernatant cake includes microbubbles having greater sizes than the target microbubbles. At 120, the first supernatant cake is removed. At 130, a second centrifugal field, which has a strength greater than the first field strength, is applied to the first supernatant cake for a second duration of time, thereby forming a second supernatant cake and a second infranatant. The second supernatant cake includes at least a portion of the target microbubbles, and the second infranatant includes microbubbles having a smaller size than the target microbubbles. At 140, the second supernatant cake is isolated.

The polydisperse microbubbles can be fabricated by any suitable methods. For example, in some embodiments, they can be obtained via mechanical agitation, e.g., by vigorous mixing of a surfactant and/or lipid containing aqueous solution while introducing gas into the suspension, or sonicating a surfactant and/or lipid containing aqueous solution. Accordingly, the polydisperse microbubbles can be coated with lipids, such as phospholipids, with surfactants, such as polymeric surfactants, with proteins, such as albumin, or with any mixtures of the above, among other materials suitable for forming microbubbles. It is noted that polymer coated microbubbles are typically more stable in a high shear stress field, such as a large centrifugal field, but they can be more difficult to prepare. Mixing a small amount of surfactants, such as polymeric nonionic surfactants, with lipids can facilitate the formation of the microbubbles. The core of the polydisperse microbubbles can include air or other gas, such as perfluorobutane, that is suitable for the intended application of the microbubbles. The gas can be incorporated into the core by a gas introducer, such as a gas tube, a sonication tip, or any other suitable means.

The polydisperse microbubbles can have a broad size distribution. The distribution depends on the coating materials of the microbubbles, the methods by which they are initially fabricated, and the condition and/or term of their storage, among other factors. For example, in some embodiments of the disclosed subject matter, the size distribution of the polydisperse microbubbles include a range from submicron, e.g., about 0.2 μm or 0.5 μm in diameter, up to more than about 10 μm in diameter, or more than about 25 μm in diameter, or even larger.

The size distribution of polydisperse microbubbles (or any size-isolated subpopulation thereof) can be characterized by measurement systems that report relative weight of each size channel (which is also referred to as size class, i.e., a very narrow band or “slice” of the size distribution spectrum, for example, a 0.1 or 0.2 μm size interval in a spectrum spanning from, e.g., 0.2 to 20 μm) of microbubbles based on the relative numbers of microbubbles falling in such size channel, for example, by methods that are rooted on light scattering principles. Alternatively, the distribution can be characterized by measurement systems that report relative weight of each size channel of microbubbles based on the relative volumes of microbubbles falling in such size channel, for example, using an electrozone sensing method. Additionally, size characterization can be performed based on fluorescence intensity and optical microscopy.

The target microbubbles have a predetermined size range that is encompassed in the size spectrum of the polydisperse microbubbles. Such a size range can be determined based on the intended use of the target microbubbles. Alternatively, such a size can be determined based on the size distribution of the polydisperse microbubbles. Accordingly, in one embodiment, the above method further includes, prior to applying any centrifugal field, obtaining a size distribution of the polydisperse microbubbles and then selecting a size range from such size distribution as the size range of the target microbubbles.

The polydisperse microbubbles can have a multimodal size distribution, i.e., the distribution contains two or more distinct “peaks” representing a relatively greater weight than other size ranges with similar widths in the entire size spectrum. The multimodal size distribution can be detected using any size characterization techniques, such as those described above. With a knowledge of the features of the multimodal size distribution, e.g., the shapes of the distribution peaks, their positions in the entire size spectrum and their relative positions to each other, appropriate adjustments to the isolation method can be made to improve the yield of the target microbubbles or to increase the efficiency of the isolation method, as will be explained below.

The centrifugal fields in the presently described techniques can be produced by any suitable one or more centrifuge devices, for example, a common bench top centrifuge, such as a bucket-rotor centrifuge having a wide range of spin speeds so as to provide a wide range of centrifugal field strengths. The centrifuge has a central axis located on the spinning axis of the rotor. The field strength of a centrifugal field can be measured by Relative Centrifugal Force (RCF), which denotes the maximum centrifugal acceleration afforded by the centrifuge at a given corresponding spinning speed (e.g., RPM) of the centrifuge rotor. To perform centrifugation, a suspension of the polydisperse microbubbles, or any subsequent infranatant formed, can be placed in a suitable container (herein generally referred to as a flotation column), such as a tube or a syringe. For convenience of operation, a (modified) syringe can be used as the flotation column. The syringe can be tubular and have a longitudinal axis and a length. Additionally, the syringe can have a cap or plunger installed on the top portion that prevents the larger buoyant microbubbles from escaping the syringe (so that they will form a supernatant cake which rests against the cap), and a drainage portion at the bottom to facilitate removal of the infranatant formed as the result of a centrifugation. The drainage portion can be positioned further away from the center axis of the centrifugal field relative to the cap. The syringe can be placed horizontally, which can provide the maximum field strength at a given spin speed, but can also be placed at a certain angle with respect to the axis of the centrifugal field.

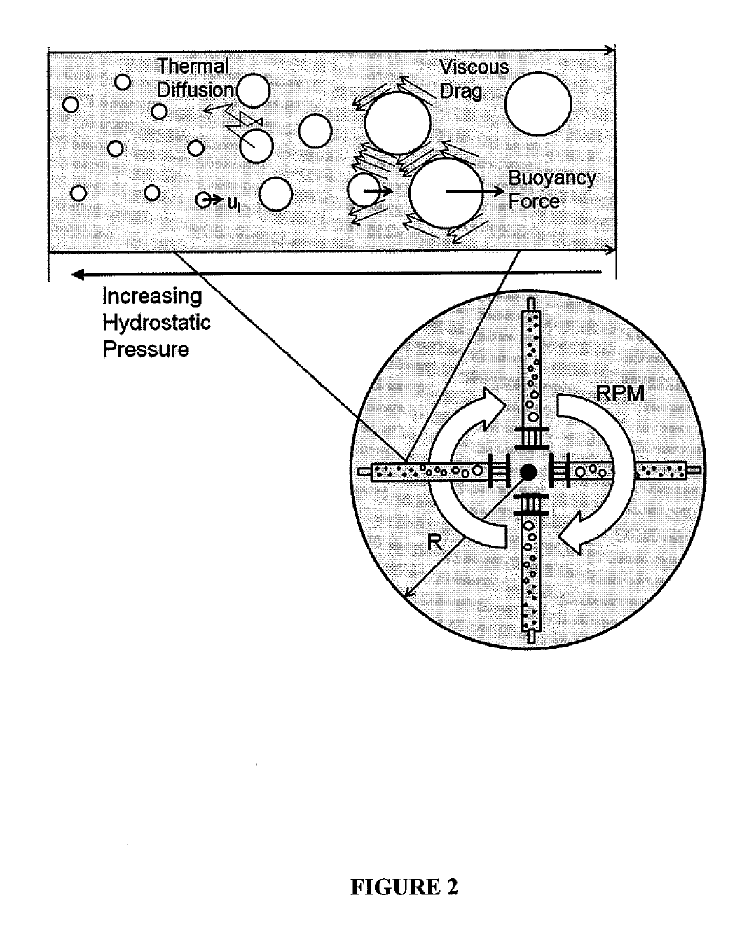

The presently described techniques for isolating target microbubbles are based in part on the principles of differential centrifugation where species of particles having different sizes and/or densities are separated in a centrifugation field as a result of their different migration speed in the centrifugal field, as illustrated in FIG. 2.

Determining the appropriate field strength for each of the multiple parts of the size fractionation process according to the disclosed subject matter can be based on a Stoke's equation, which describes the rise velocity of a buoyant particle relative to the bulk fluid under creeping flow conditions as

$${u}_{i} = \frac{2 ( {ρ}_{2} - {ρ}_{1 i} )}{9 {η}_{2}^{*}} {r}_{i}^{2} {g}_{c} , \qquad ( 1 )$$

where subscript i refers to the particle size class, rᵢ is the particle radius, ρ₂ is the density of the liquid medium where the microbubbles are suspended, ρ₁ᵢ is the density of the particle, and g_{c} is the centrifugal acceleration measured in RCF. (g_{c}=RCF×g, where g is the acceleration in a gravitational field, i.e., 9.8 m/s²). The effective viscosity, η₂*, of the microbubbles suspension can be calculated using Batchelor and Greene's correlation for the modified fluid viscosity:

$$\begin{matrix}\frac{{η}_{2}^{*}}{{η}_{2}} = 1 + 2.5 Φ + 7.6 {Φ}^{2} , & ( 2 ) \\ Φ = {\sum }_{i = 1}^{{N}_{d}} {Φ}_{i} , & ( 3 )\end{matrix}$$

where η₂ is the viscosity of the suspension medium, and Φ is total the microbubble volume fraction for N_{d} size classes. Equations 1-3 can be used to calculate the strength of the centrifugal field (in RCF) for a given initial size distribution of the polydisperse microbubbles, time period and the length of the flotation column. Volume fraction can be assumed to be constant over the entire column, and acceleration/deceleration effects can be neglected. It is noted that the above Stoke's equation yields more accurate results when the volume fractions of the microbubbles in a suspension subjected to the centrifugal field are equal to or below 20%. A larger volume fraction of microbubbles would cause more significant collision among the migrating particles and accordingly more turbulence in the suspension, thereby making the creeping flow condition assumption less true.

In one embodiment, the appropriate field strengths to be used in the first centrifugal field and the second centrifugal field can be obtained as follows. First, the size distribution of the initial polydisperse microbubbles is measured. The size distribution is then imported into a spreadsheet in order to determine the number density for each size class and the total gas volume fraction. The spreadsheet is used to calculate the relative centrifugal force (RCF) needed for a microbubble size class to rise through the flotation column of length L for a predetermined centrifugation time. The requisite field strengths thus obtained can be tabulated before performing the first centrifugation, and serve as a convenient guide for selecting the appropriate spinning speed (RPM) of the centrifuge to remove fractions of microbubbles larger than the target microbubbles or remove fractions of microbubbles smaller than the target microbubbles.

The duration of time for any applied centrifugal field can be predetermined. For example, a duration of time of 1 minute can be chosen for all the centrifugal fields that may need to be applied in order to isolate the target microbubbles. Other values of the duration of time can also be used, for example, 0.5 minutes, 2 minutes, 4 minutes, or any other suitable lengths of time. The duration of time for each of centrifugal field does not need to be the same. For example, a first centrifugal field can be applied for 2 minutes, and a second centrifugal field can be applied for 1 minute. The duration of times can be chosen to be longer than 1 minute in order to minimize transient effects caused by the acceleration and deceleration of the rotor, so that the sample experiences mostly a constant centrifugal field strength.

The suitable field strength of an applied centrifugal field in the described techniques depend on the coating material of the microbubbles, the size distribution of the microbubbles, and the length of the flotation column, among others, and can be between 1 and 500 equivalent gravity. Greater field strength may result in significant microbubble destruction, thereby lowering the yield of the target microbubbles. This range of field strengths can be applied to microbubbles dispersion containing microbubbles in the 1-10 μm diameter range. The specific field strength values can be determined empirically or estimated either by Stokes Law, or through empirical measurements when size measurement is available. The preferred method is to use Stokes Law as an initial estimate and the refine the technique through empirical measurement. One can use Stokes law based on the initial size distribution of the polydisperse microbubbles as outlined above, or a priori without knowledge of the initial size distribution or concentration. For example, one can measure the volume and mass of the microbubble suspension and, based on a comparison of the density of the suspension medium with microbubbles with the density of the suspension medium without microbubbles (i.e., before microbubbles are generated), one may determine the volume fraction of encapsulated gas in computing η₂*.

Obtaining a size distribution of the initial polydisperse microbubbles, prior to subjecting the microbubbles to a centrifugal field, can be useful in a multiple ways:

(1) Selecting a proper strength of a centrifugal field to be applied from the size distribution given a desired or predetermined duration of time during which such a field is to be applied. For example, if one desires to remove all the microbubbles having a greater size than the target microbubbles in one centrifugation, the RCF needed for removing such fractions (as supernatant cake) can be computed based on the above Stoke's equation given a predetermined time period during which the centrifugal field is to be applied.

(2) Validating the initial polydisperse microbubbles. For example, through an examination of the shape of the size distribution, one can determine whether the target microbubbles are present in a sufficient amount in the polydisperse microbubbles to warrant further processing (otherwise the yield of the target microbubbles would not be sufficient for the intended applications). If not, another sample of the polydisperse microbubbles can be obtained and checked for size distribution.

(3) Improving efficiency of the size-isolation process. Through an examination of the shape of the size distribution, one can identify the shape and position of possible peak(s) in the distribution. For example, if the target microbubbles have a range of about 4 to 5 μm, and the size distribution has a rather focused peak in that range and only a minor tail on the positive side of 5 μm, e.g., a small tail in 5-6 μm range, one can use a centrifugal strength that is only sufficient to remove the fractions of microbubbles having a larger size than 6 μm (instead of a greater centrifugal strength that would remove fractions of microbubbles having a size of larger than 5 μm). This can still obtain a sufficiently monodisperse target microbubbles without suffering from the substantial loss of the target microbubbles, because a greater centrifugal field (sufficient to remove the microbubbles having a size of 5 μm and above) would cause a greater portion of the target microbubbles to collect into the supernatant cake (which will be discarded).

(4) Facilitating the design of a multiple part fractionation process for microbubbles having sizes larger than the target microbubbles. When the initial polydisperse microbubbles have a broad distribution on the positive side of the target microbubbles, a multiple fractioning process can achieve higher isolation efficiency. For example, if the target microbubbles have a range of 1 to 2 μm and the size distribution of the initial polydisperse microbubbles have a significant profile in the range of greater than 2 μm, one can use a first gentle centrifugal field to remove the fractions of microbubbles having a size of greater than 10 μm, and then use a second centrifugal field (stronger than the first one) on the remaining infranatant to remove the fractions of microbubbles having a size of greater than 7 μm. Likewise, a third centrifugal field (stronger than the second one) can be used on the second infranatant to remove the fractions of microbubbles having a size of greater than 4 μm, and so on, until substantially all the microbubbles having a size larger than 2 μm are removed. Such a multiple part size-fractionation process essentially multiplies the length of the flotation column, and therefore can achieve a better separation of different sizes of microbubbles and a higher yield of the target microbubbles as compared with a single centrifugation using a large field strength.

A size distribution can also be obtained between the two successive centrifugations to validate the result of the first centrifugation. If necessary, a centrifugation can be repeated to improve the result. Besides, the infranant(s) that contains the target microbubbles which is subject to further centrifugation(s) can be diluted (or redispersed) to the maximum volume allowed in the flotation column before applying a subsequent centrifugation, thereby ensuring that the full length of the flotation column can be utilized to allow a more effective separation of different sizes of microbubbles.

After substantially all the microbubbles having a greater size than the target microbubbles are removed in one or more parts of the process as outlined above (in the form of one or more supernatant cakes), a further centrifugation of the infranatant containing the target microbubbles can be performed using a field strength and duration of time sufficient to cause substantially all of the target microbubbles to form a supernatant cake. Depending on the size distribution of the polydisperse microbubbles, this cake can contain an amount of microbubbles having a size smaller than the target microbubbles that render the cake not as monodisperse as desired. If such is the case, the cake can be further purified. For example, the cake containing the target microbubbles can be redispersed into a new dispersion, and a further centrifugation can be performed on the new dispersion. For quickly improving the purity of the cake (at the expense of a lower yield of target microbubbles), the centrifugal strength for the purification can be selected as approximately the same as or slightly smaller than the one used in the previous step (i.e., the one used to collect substantially all of the target microbubbles to form the supernatant cake). Likewise, the duration of time applied for purification can be selected as similar or slightly smaller than the duration of time last used. The purification can be repeated until an end point is reached, for example, a size distribution of the isolated cake containing the target microbubbles satisfying the needs of the intended application, or by simple visual inspection of the infranant(s) formed, e.g., when the infranant(s) is no longer turbid, indicating the smaller-sized microbubbles are removed from the target microbubbles to a satisfactory degree.

In some embodiments of described techniques of the disclosed subject matter, the initial polydisperse microbubbles have a size range of about 0.5 μm to about 10 μm. In one embodiment, the target microbubbles have a size range of about 4 to about 5 μm. In such an embodiment, a first applied centrifugal field can have a strength of about 70 RCF to remove microbubbles having a size larger than 6 μm, and a second applied centrifugal field can have a strength of about 160 RCF to remove the microbubbles having a size smaller than 4 μm. In another embodiment, the target microbubbles have size range of about 1 μm to about 2 μm. In such an embodiment, a first applied centrifugal field can have a strength of about 270 RCF to remove microbubbles having a size larger than 2 μm, and a second applied centrifugal field can have a strength of about 300 RCF to remove the microbubbles having a size smaller than 1 μm.

Depending on the particular processes performed and parameters selected according to the techniques of the disclosed subject matter, the isolated final cake containing the target microbubbles can attain a purity of greater than about 80%, 90%, 95%, 99%, or even higher, meaning that the fractions of microbubbles falling out of the predetermined size range of the target microbubbles can be lower than about 20%, 10%, 5%, 1%, or even lower by volume. In addition, the polydispersity of the target microbubbles can achieve about 0.2 μm, 0.1 μm, or lower in polydispersity index (PI), which is defined as the volume-weighted mean diameter divided by the number-weighted mean diameter. These microbubbles with a narrow size distribution are also referred to as monodisperse microbubbles.

In another aspect of the disclosed subject matter, a method for performing high frequency ultrasonic imaging is disclosed. The method includes administering a suspension of microbubbles having a predetermined size range to an animal, and performing the ultrasonic imaging on the animal, e.g., on an organ of the animal, at a fundamental frequency of at least about 30 MHz. The animal can be a mammal, for example, a mouse, a rabbit, a human, or any other suitable mammal. The organ can be an internal organ, for example, a kidney, a liver, and any other suitable organs. The microbubbles can be monodisperse, and can have a size range of, for example, about 1-2 μm, about 4-5 μm, or about 6-8 μm. Microbubbles having different size ranges can be used for different application. For example, microbubbles having a size of about 1-2 μM can be used where negative contrast and shadowing are desired, e.g., for reperfusion studies of very fine capillaries; microbubbles having a size of about 6-8 μm can be used where a large positive contrast and less shadowing are desired.

As used herein, the term “about” means that the deviation of the quantity modified by the term can have no more than 10% from the quantity specified or predetermined; in the absence of a specified or predetermined value, the term means that the relative standard deviation of multiple measurements of the same quantity does not exceed 10% of the average of the multiple measurement results.

As used herein, the term “substantially all” means that the portion of the objects modified by this term should be at least 75% of all such objects, and more preferably 85%, 90%, or 95% of all such objects.

As used herein, the term that “have [having] a size range” means that the microbubbles consist of at least 80% of microbubbles in the specified range, and preferably consist of at least 90%, 95%, or 99% of microbubbles in the specified range.

As used herein, all mention of sizes of any microbubbles refers to the diameter of the microbubbles.

### EXAMPLES

The following examples are merely illustrative of the presently described subject matter and should not be considered as limiting the scope of the disclosed subject matter in any way.

### Example 1

### Isolation of Microbubbles Having a Predetermined Range of 1-2 Microns and 4-5 Microns and Characterizations Thereof

### Materials and Methods

### Materials

All solutions were prepared using filtered, 18 MΩ deionized water (Direct-Q, Millipore, Billerica, Mass.). All glassware was cleaned with 70 vol % ethyl alcohol solution (Sigma-Aldrich; St. Louis, Mo.) and rinsed with deionized water. The gas used to form microbubbles was perfluorobutane (PFB) at 99 wt % purity obtained from FluoroMed (Round Rock, Tex.). All phospholipids were purchased from Avanti Polar Lipids (Alabaster, Ala.) and initially dissolved in chloroform (Sigma-Aldrich) for storage. Polyoxyethylene-40 stearate (PEG40S) was obtained from Sigma-Aldrich and dissolved in deionized water. The fluorophore probe 3,3′-dioctadecyloxacarbocyanine perchlorate (DiO) solution (Invitrogen; Eugene, Oreg.) was used to label the microbubbles for part of the experiments.

### Generation of Microbubbles

Microbubbles were coated with DSPC and PEG40S at molar ratio of 9:1. The indicated amount of DSPC was transferred to a separate vial, and the chloroform was evaporated with a steady nitrogen stream during vortexing for about ten minutes followed by several hours under house vacuum. 0.01 M phosphate buffered saline (PBS) solution (Sigma-Aldrich) was filtered using 0.2-μm pore size polycarbonate filters (VWR, West Chester, Pa.). The dried lipid film was then hydrated with filtered PBS and mixed with PEG40S (25 mg/mL in filtered PBS) to a final lipid/surfactant concentration of 1.0 mg/mL. The lipid mixture was first sonicated with a 20-kHz probe (model 250A, Branson Ultrasonics; Danbury, Conn.) at low power (power setting dialed to 3/10; 3 Watts) in order to heat the pre-microbubble suspension above the main phase transition temperature of the phospholipid (˜55° C. for DSPC) and further disperse the lipid aggregates into small, unilamellar liposomes. PFB gas was introduced by flowing it over the surface of the lipid suspension. Subsequently, higher power sonication (power setting dialed to 10/10; 33 Watts) was applied to the suspension for about 10 seconds at the gas-liquid interface to generate microbubbles. For flow cytometry and fluorescence microscopy experiments, DiO solution (1 mM) was added prior to high-power sonication at an amount of 1 μL DiO solution per mL of lipid mixture.

### Microbubble Washing & Lipid Recycling

The microbubble suspension was collected into 30-mL syringes (Tyco Healthcare, Mansfield, Mass.), which were used as the flotation columns. Washing and size fractionation by centrifugation was performed with a bucket-rotor centrifuge (model 5804, Eppendorf, Westbury, N.Y.), which had a radius of approximately 16 cm from the center to the syringe tip and operated between 10 and 4500 RPM. Centrifugation (10 minutes, 300 RCF) was performed to collect all microbubbles from the suspension into a cake resting against the syringe plunger. The remaining suspension (infranatant), which contained residual lipids and vesicles that did not form part of the microbubble shells, was recycled to produce the next batch of microbubbles. All resulting cakes were combined and re-suspended in PBS to improve total yield.

### Size and Concentration Measurements

Microbubble size distribution was determined by laser light obscuration and scattering (Accusizer 780A, NICOMP Particle Sizing Systems, Santa Barbara, Calif.). 2-μL samples of each microbubble suspension were diluted into a 30-mL flask under mild mixing during measurement. Size distribution was also determined using the electrozone sensing method (Coulter Multisizer III, Beckman Coulter, Opa Locka, Fla.). A 4-4 sample of microbubble suspension was diluted into a 60-mL flask and stirred continuously to prevent flotation-induced error. A 30-μm aperture (size range of 0.6-18 μm) was used for the measurements. All samples were measured at least three times by either instrument and analyzed for both number- and volume-weighted size distribution.

### Optical Microscopy

Direct visual confirmation of microbubble size was performed 48 hours after the samples were prepared using an Olympus 1×71 inverted microscope (Olympus; Center Valley, Pa.). The microbubble samples were taken directly from the serum vials and imaged at room temperature. Images were captured in both bright-field and epi-fluorescence modes using a high-resolution digital camera (Orca HR, Hamamatsu, Japan) and processed with Simple PCI software (C-Imaging, Cranberry Township, Pa.). A 40× objective was used to capture the images of size-isolated microbubbles of 4-5 μm diameter, while a 100× oil-immersion objective was used for polydispersed microbubbles and size-isolated microbubbles of 1-2 μm diameter. Subsequent image analysis was done using ImageJ 1.4 g (available at National Institute of Health website).

### Flow Cytometry

A FACScan Cell Analyzer (Becton-Dickinson, Franklin Lakes, N.J.) was used to characterize microbubble fluorescence intensity (FL) and light scattering profiles (FSC and SSC). Voltage and gain settings for FSC, SSC and FL were adjusted to delineate the microbubble populations from instrument and sample noise. 10 μL samples were diluted with 3 mL deionized water prior to each measurement. Subsequent data analysis was done using CellQuest Pro (Becton-Dickinson, Franklin Lakes, N.J.).

### Size Isolation

Following production, microbubbles were collected into 30-mL syringes (length=8.2 cm) and washed, as above. Production-washing was repeated 3-5 times, each time saving the microbubble cake and recycling the lipid infranatant. The cakes were combined and re-dispersed into 30 mL of filtered PBS. In order to ensure a high yield, the concentration of microbubbles after such re-dispersion should be at least ˜1 vol %. Microbubbles of 1-2 μm and 4-5 μm diameter are isolated as follows. At least three separate experimental runs were performed for each isolation, and size distributions were measured at least three times each.

### Isolation of 4-5 μm Diameter Microbubbles

Before beginning the isolation process, care was taken to remove large, visible bubbles that can form during production or subsequent handling. Microbubbles of greater than 10-μm diameter were removed by performing one centrifugation cycle at 30 RCF for 1 min. The infranatant consisting of less than 10-μm diameter microbubbles was saved and re-dispersed in 30 mL PBS, while the cake was discarded. Next, microbubbles of greater than 6-μm diameter were removed by performing one centrifugation cycle at 70 RCF for 1 min. The infranatant consisting of less than 6-μm diameter microbubbles was saved and re-dispersed to 30 mL PBS; the cake was discarded. Finally, microbubbles of less than 4-μm diameter were removed by centrifuging at 160 RCF for 1 min. This was repeated about 5-10 times, while each time the infranatant was discarded and the cake was re-dispersed in filtered PBS. Alternatively, 12-mL syringes (L=6.3 cm) were employed and centrifuged at 120 RCF for 1 min to improve yield. These cycles were repeated until the infranatant was no longer turbid, indicating complete removal of microbubbles less than 4 μm. The final cake was concentrated to a 1-mL volume of 20 vol % glycerol solution in PBS and stored in a 2-mL serum vial with PFB headspace.

### Isolation of 1-2 μm Diameter Microbubbles

The infranatant collected from the 4-5 μm isolation was centrifuged at 270 RCF for 1 min for one cycle in order to remove microbubbles of approximately 3-μm diameter and above by collecting them into the cake. The infranatant consisted mostly of microbubbles 1-2 μm diameter. The target microbubbles were collected into a concentrated cake by centrifuging at 300 RCF for 10 min. The final cake was re-dispersed to a 1-mL volume of 20 vol % glycerol solution in PBS and stored in a 2-mL serum vial with PFB headspace.

### Results

### Polydispersity of Freshly Sonicated Microbubbles

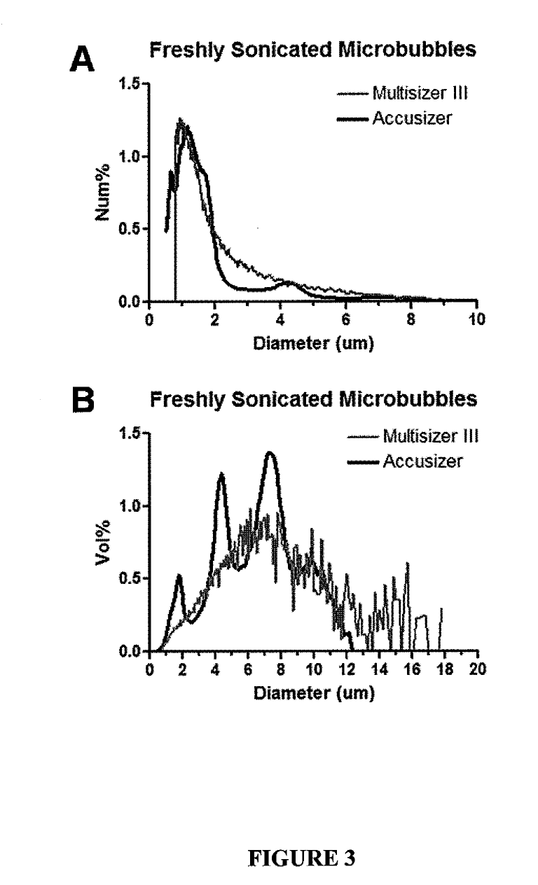

Preparation of microbubbles by sonication of a 50 mL lipid mixture resulted in a polydisperse suspension of approximately 10⁹ to 10¹⁰ particles mL⁻¹. Particle sizing with the Accusizer and Multisizer showed a distribution ranging from the lower limit of resolution, ˜0.5 μm, to greater than 15 μm diameter (FIG. 3). A significant portion of the freshly generated suspension contained submicron microbubbles. Submicron microbubbles also have been observed by static light scattering and freeze-fracture transmission electron microscopy. For larger microbubbles, the number-weighted distribution tailed off near 6-8 μm diameter (FIG. 3A). The volume-weighted distribution, however, showed a significant population out to greater than 10 μm diameter (FIG. 3B). As volume is directly proportional to the cube of the diameter, microbubbles with larger diameters tended to skew the volume-weighted size distribution. Median volume-weighted diameters were chosen in order to evaluate the samples during size isolation, since this gives a more rigorous indication of the central tendency than arithmetic mean in a skewed distribution.

Interestingly, the Accusizer consistently measured distinct peaks centered on approximately 1-2, 4-5, 7-8 and 9-11 μm diameter for each batch of lipid-coated microbubbles. These peaks were clear from the volume-weighted distribution, but they also could be discerned from the number-weighted distribution. In the laboratory, these peaks were observed for a variety of gas and lipid combinations (data not shown). Size distribution was also measured using a Multisizer III. While the Accusizer measures size based on light obscuration and scattering, the Multisizer utilizes electrical impedance sensing of the volume of electrolyte displaced by the microbubble as it passes through an orifice. The multimodal distribution was not observed on the Multisizer, which gave a broad distribution with a single peak located at ˜1 μm for the number-weighted distribution and ˜8 μm for the volume-weighted distribution. From this data, it was unclear whether the multimodal distribution was real, and could not be resolved by the Multisizer, or if it was an artifact of the Accusizer. Therefore an improved characterization of the microbubble distribution was sought.

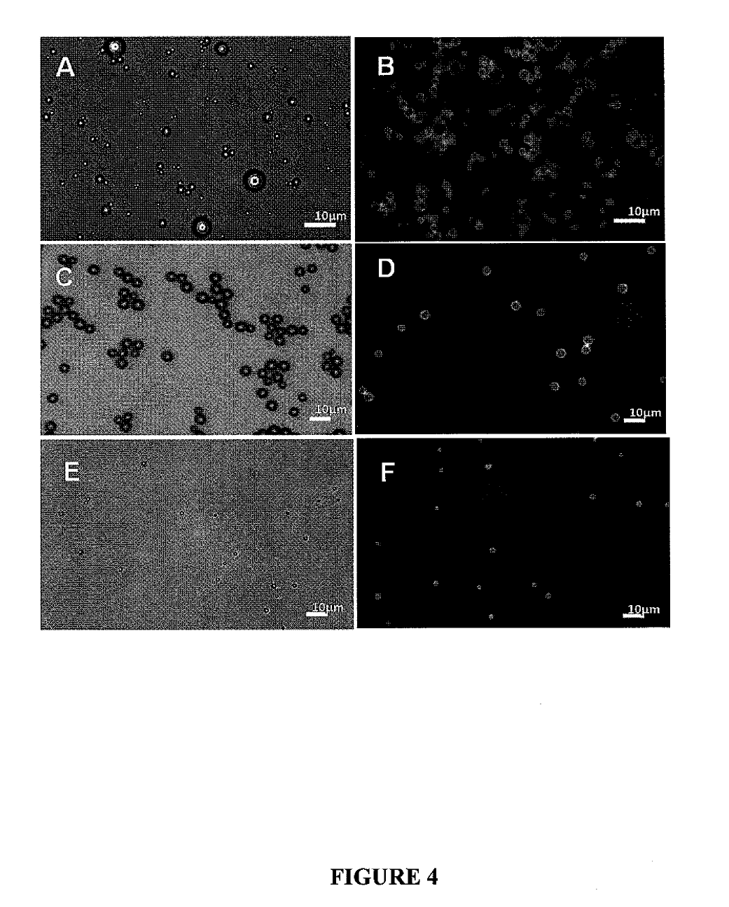

Microscopy allowed direct visual inspection of individual microbubbles from the suspension. Bright-field and epi-fluorescence microscopy images are shown in FIG. 4 (Bright-field images (left) and fluorescence images (right) of the membrane probe DiO; Arrows point to microstructural features of high probe density. The initial, polydisperse microbubble suspensions (A, B) are shown for comparison to the isolated 4-5 μm diameter microbubbles (C, D) and 1-2 μm diameter microbubbles (E, F). Analysis of the bright-field images using ImageJ gave mean diameters of 4.6±0.3 μm and 1.8±0.3 μm for microbubbles seen in FIGS. 4C and 4E, respectively). In fluorescence mode, microbubbles appeared as bright rings with dark centers, clearly showing uptake of DiO into the shell. In bright-field mode, microbubbles appeared as dark spheres with bright centers. Diffraction rings were particularly prevalent for the smaller microbubbles. This confirmed the predominance of gas-filled microbubbles in the suspension. Analysis of the bright-field images using ImageJ indicated that the distribution of the freshly generated microbubbles was multimodal, with a mean diameter of 4.0±3.0 μm for the image shown in FIG. 4A.

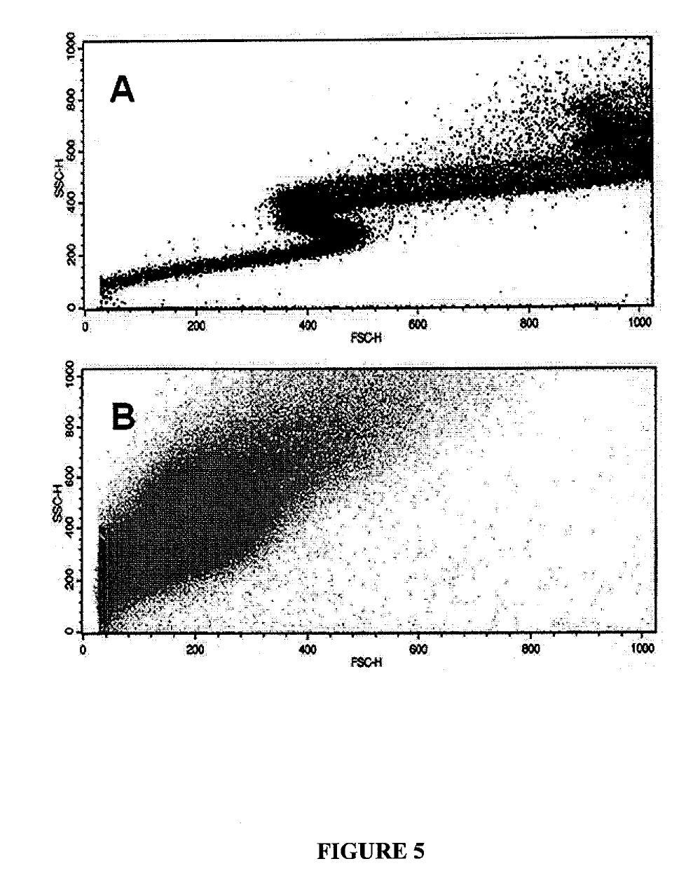

Flow cytometry was used to further characterize the polydisperse microbubbles, as shown in FIG. 5 (FIG. 5(A) shows the serpentine trend for the initial polydisperse microbubble suspension (cytometer settings: detector P1, voltage E01, amp 1.98; detector P2, voltage 287, amp 1.49); FIG. 5(B) shows no serpentine trend for the isolated 4-5 μm microbubbles (cytometer settings: detector P1, voltage E01, amp 2.30; detector P2, voltage 173, amp 2.88)). Forward-(FSC) and side-(SSC) light scattering measurements were taken. A serpentine shape was observed on the dot plot of FSC versus SSC for the polydisperse microbubble suspensions, as shown in FIG. 5A. The serpentine shape appeared to correlate with the distinct peaks found by the Accusizer.

The origins of polydispersity in the freshly generated suspension of lipid-coated microbubbles observed here can be explained by the multiple interacting mechanisms occurring during entrainment and cavitation-induced disintegration, as described above. The fact that the microbubbles themselves can be oscillating in the acoustic field and can act as cavitation nuclei adds further complexity to analysis. Additionally, the dynamics of lipid adsorption and spreading and monolayer shell formation are expected to play a role in determining the “apparent surface tension” and, for the lipids used here, can be expected to add additional surface viscosity and elasticity terms. While polydispersity can be unavoidable, the ability of mechanical agitation to rapidly generate large numbers of microbubbles brings this technique to the forefront of current microbubble creation methods. Given the excellent stability of lipid-coated microbubbles and the apparent presence of distinct peaks in the multimodal distribution, size isolation by differential centrifugation appeared to be a feasible approach. In the following, experiments for isolating narrow distributions of target microbubbles and characterization of their size distribution and long-term stability are described.

### Size Isolation of Microbubbles

Submicron microbubbles were found to be relatively unstable and therefore were not isolated. Instead, microbubbles in the 1-2 μm and 4-5 μm diameter ranges were isolated. These 15′ ranges are interesting for biomedical applications. While both sizes are comparable to that of an erythrocyte, they can yield different biodistribution, resonance frequencies, and acoustically induced bioeffects. In general, the 1-2 μm microbubbles were approximately 100-fold more abundant than the 4-5 μm microbubbles in the initial dispersion.

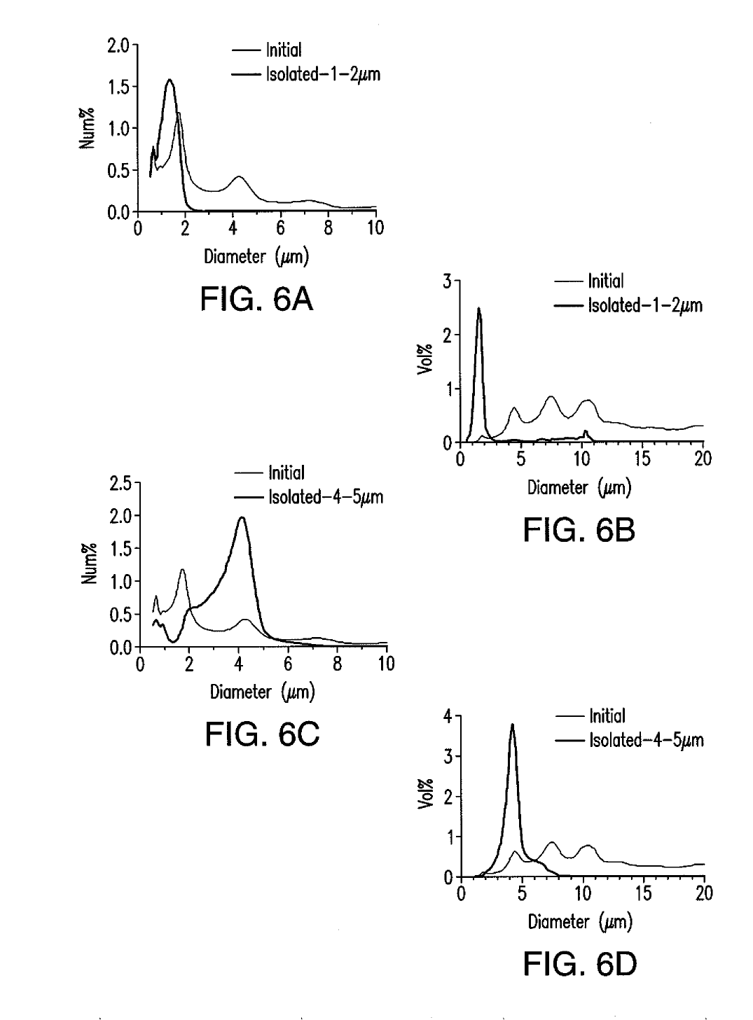

Microbubbles in the larger diameter range (4-5 μm) were isolated first, while the smaller microbubbles were saved for the subsequent isolation of the 1-2 μm fraction. After repeated centrifugation and re-concentration according to the simple model, microbubbles with diameters of 4-5 μm were successfully isolated from the initial polydisperse suspension, as shown in FIG. 6 (shown are isolated subpopulations at the 1-2 μm (A, B) and 4-5 μm (C, D) diameter size ranges; results are summarized in Table 1).

TABLE 1
Summary of size distributions.

| | Total | Number-Weighted | | Volume-Weighted | | |
|---|---|---|---|---|---|---|
| | Concen- | Diameter (μm) | | Diameter (μm) | | |
| | tration | (Mean ± | (Median ± | (Mean ± | (Median ± | |
| Sample | (#/mL) | SD) | SD) | SD) | SD) | PI |
| Initial | 1.35E+10 | 2.1 ± | 1.4 ± | 19.4 ± | 19.0 ± | 10.5 ± |
| | | 0.5 | 0.6 | 17.1 | 17.3 | 10.6 |
| 1-2 μm | 2.8E+09 | 1.3 ± | 1.3 ± | 1.8 ± | 1.5 ± | 1.5 ± |
| Isolated | | 0.2 | 0.2 | 0.1 | 0.2 | 0.2 |
| 4-5 μm | 1.01E+08 | 2.8 ± | 3.0 ± | 4.2 ± | 4.1 ± | 1.5 ± |
| Isolated | | 0.2 | 0.2 | 0.1 | 0.1 | 0.1 |

Multiple centrifugations were needed to expel smaller microbubbles (<4 μm), which were more abundant in the initial suspension. The final 4-5 μm microbubble suspension typically had a total volume of 1 mL with concentration in the order of 10⁸ to 10⁹ mL⁻¹, as determined by the Accusizer. Table 1 summarizes both averaged number-weighted and volume-weighted mean and median values for each size fraction.

Microbubbles of 1-2 μm diameter were isolated in fewer centrifugation than for the 4-5 μm microbubbles. For instance, separation of microbubbles less than 2 μm diameter in the infranatant was typically completed by a single centrifugation. However, the final part of the process for concentrating microbubbles greater than 1-μm diameter required substantially higher centrifugal force than for the 4-5 μm microbubbles, which is consistent with their lower buoyancy. The final 1-2 μM microbubble suspension typically had a total volume of 1 mL with concentration on the order of 10⁹ to 10¹⁰ mL⁻¹, as determined by the Accusizer.

### Characterization of the Isolated Microbubbles

Table 1 gives the average PI value for the freshly generated microbubbles and the size-isolated microbubbles. The initial suspension was highly polydisperse, with PI values as high as 18 but no lower than 6. The PI for the 4-5 μm fraction was only 1.5±0.1, while that of the 1-2 μm fraction was only 1.5±0.2.

Bright-field and epi-fluorescence microscopy images provided direct visual confirmation for the narrow size distribution of size-isolated microbubbles, as shown in FIG. 4. These results are in agreement with the size distributions determined by the Accusizer and Multisizer. Fluorescence images also showed microstructural features within the lipid shell. For example, brighter spots indicating non-uniform fluorophore distribution were often observed (see arrows in FIGS. 4D and 4F).

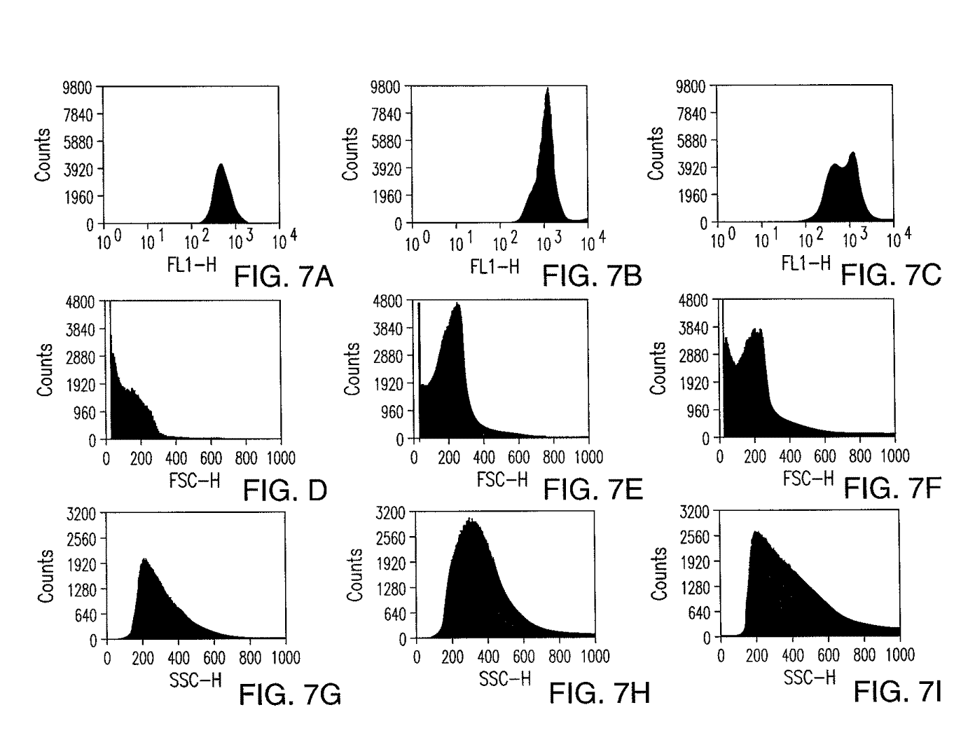

Flow cytometry was performed to characterize the size-isolated fractions, as shown in FIG. 7. (For FIG. 7, three different tests (fluorescence intensity FL, forward-FSC and side-SSC light scattering versus particle count) were performed for the same sample as represented by each column of plots. Column 1 (A, D, G) and Column 2 (B, E, H) samples of FIG. 7 had median volume-weighted diameters of 1.8 μm and 4.6 respectively. Column 3 (C, F, I) was a mixture of these two suspensions. FL measurements (A-C) confirmed their relative size distributions with median values of 500, 1114 and 792, respectively. FSC (D-F) and SSC (G-I) measurements showed a similar tend: (FSC) 131, 214 and 194; (SSC) 286, 349 and 376, respectively. Results are summarized in Table 2.)

TABLE 2
Summary of flow cytometry measurements.

| | Fluorescence | Forward-Light | Side-Light |
|---|---|---|---|
| Microbubble | Intensity | Scattering | Scattering |
| Diameter | (Median ± SD) | (Median ± SD) | (Median ± SD) |
| 1-2 μm | 509.54 ± 16.02 | 137.00 ± 5.29 | 289.33 ± 4.93 |
| 4-5 μm | 1114.33 ± 35.03 | 214.33 ± 7.37 | 417.33 ± 23.50 |
| Mixture | 775.17 ± 17.51 | 193.67 ± 0.58 | 375.67 ± 1.53 |

Fluorescence intensity (FL), FSC and SSC measurements were all taken under the same cytometer settings. The serpentine shape was not observed for the size-isolated suspensions, as it was for the polydisperse case. Instead, the data points were found to be clustered in one region of the dot plot. The lack of the serpentine shape in the size-isolated samples indicated that they were indeed subpopulations of the initial multimodal sample. Table 2 lists the median values of three cytometry tests for each microbubble sample. Comparison of the FSC and SSC results for individual, size-isolated fractions and their mixture supported the existence of two distinct microbubble subpopulations. Monomodal distributions were observed for the individual size-isolated suspensions, with a lower median value corresponding to the 1-2 μm microbubbles. When the size-isolated microbubbles were subsequently mixed together, a bimodal distribution appeared with two distinct peaks that agreed with the respective median values for the individual suspensions.

A single peak was observed on the FL histogram for the size isolated microbubbles (FIG. 7). The median FL value for 1-2 μm microbubbles was lower than that for the 4-5 μm microbubbles. Upon mixing, a bimodal distribution was observed with peak median FL values corresponding to those of the individual suspensions. Assuming that each microbubble is a perfect sphere and the fraction of fluorophores in the lipid shell is the same for all microbubbles, regardless of size, the number of fluorophores per microbubble should be directly proportional to the surface area, or the square of the diameter. This was confirmed when comparing the averaged FL values versus microbubble squared diameter. The fluorescence intensity value for the mixture of 1-2 and 4-5 μm microbubble samples (775±18) agreed with the average between the FL values measured for each individual monodisperse suspension (510±16 and 1110±35).

In brief, the above results demonstrated the effectiveness of the isolation methods for isolating distinct fractions of the microbubbles at the desired size ranges.

### Stability of Size-Isolated Microbubbles

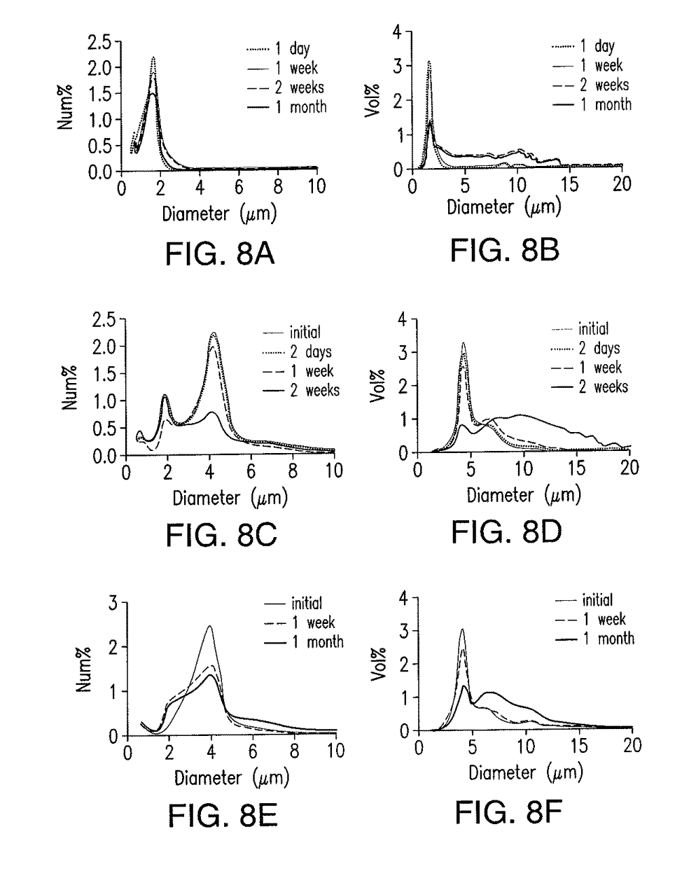

For biomedical applications, it is desired that the microbubbles be stable for at least 48 hours at their respective size distributions. A test of microbubble stability was performed using samples concentrated to 10¹° mL⁻¹ for 1-2 μm microbubbles, and 10⁸ mL⁻¹ or 10⁹ mL⁻¹ for 4-5 μm microbubbles, in a 1-mL volume of 20 vol % glycerol in PBS and stored in a sealed 2-mL serum vial with PFB headspace, as shown in FIG. 8. (FIG. 8 shows the distributions at various time points for the 1-2 μm (A, B) and 4-5 μm (C, D, E, F) diameter microbubbles. Number-weighted (A, C, E) and volume-weighted (B, D, F) distributions are shown for inspection of polydispersity. The suspensions initially were dispersed in 1-mL volume of PBS with 20 vol % glycerol, with a concentration of ˜10¹⁰ mL⁻¹ for the 1-2 μm diameter microbubbles and either ˜10⁸ mL⁻¹ (C, D) or ˜10⁸ mL⁻¹ (E, F) for the 4-5 μm diameter microbubbles. Each curve is the average of three experiments with three measurements each. Results are summarized in Table 3.)

TABLE 3
Summary of stability of size-isolated microbubble suspensions.

| | | | Number Weighted | | Volume Weighted | | |
|---|---|---|---|---|---|---|---|
| | Total | Volume | Diameter (μm) | | Diameter (μm) | | |
| | Concentration | Fraction | (Mean ± | (Median ± | (Mean ± | (Median ± | |
| Time | (#/mL) | (mL/mL) | SD) | SD) | SD) | SD) | PI |
| | 1-2 micron bubble stability | | | | | | |
| Initial | 2.28E+10 | 3.740% | 1.3 ± 0.1 | 1.2 ± 0.1 | 1.9 ± 0.1 | 1.8 ± 0.1 | 1.5 ± 0.1 |
| 1 day | 8.25E+09 | 1.096% | 1.2 ± 0.1 | 1.2 ± 0.1 | 1.7 ± 0.1 | 1.6 ± 0.1 | 1.4 ± 0.1 |
| 7 days | 7.60E+09 | 1.308% | 1.3 ± 0.1 | 1.4 ± 0.1 | 1.8 ± 0.1 | 1.7 ± 0.1 | 1.4 ± 0.1 |
| 14 days | 4.31E+09 | 0.787% | 1.4 ± 0.1 | 1.4 ± 0.1 | 2.0 ± 0.1 | 1.7 ± 0.1 | 1.4 ± 0.1 |
| 28 days | 1.12E+09 | 0.388% | 1.5 ± 0.1 | 1.4 ± 0.1 | 4.1 ± 0.1 | 2.6 ± 0.1 | 2.8 ± 0.1 |
| | 4-5 micron bubble stability for suspensions containing less than 1% gas | | | | | | |
| | volume | | | | | | |
| Initial | 1.18E+08 | 0.340% | 3.2 ± 0.1 | 3.4 ± 0.1 | 4.8 ± 0.1 | 4.5 ± 0.1 | 1.5 ± 0.1 |
| 2 days | 1.04E+08 | 0.294% | 3.2 ± 0.1 | 3.4 ± 0.1 | 4.8 ± 0.1 | 4.4 ± 0.1 | 1.5 ± 0.1 |
| 7 days | 1.01E+08 | 0.305% | 3.1 ± 0.1 | 3.2 ± 0.1 | 5.3 ± 0.1 | 4.6 ± 0.1 | 1.7 ± 0.1 |
| 14 days | 3.87E+07 | 0.148% | 2.8 ± 0.1 | 2.1 ± 0.1 | 8.1 ± 0.1 | 7.6 ± 0.1 | 3.0 ± 0.1 |
| | 4-5 micron bubble stability on suspensions containing greater than 1% gas | | | | | | |
| | volume | | | | | | |
| Initial | 6.30E+08 | 1.890% | 3.3 ± 0.1 | 3.5 ± 0.1 | 4.9 ± 0.1 | 4.3 ± 0.1 | 1.5 ± 0.1 |
| 7 days | 5.01E+08 | 1.090% | 2.9 ± 0.1 | 2.9 ± 0.1 | 4.8 ± 0.3 | 4.1 ± 0.1 | 1.6 ± 0.1 |
| 28 days | 2.98E+08 | 1.000% | 3.1 ± 0.1 | 3.0 ± 0.1 | 6.2 ± 0.1 | 6.0 ± 0.1 | 2.0 ± 0.1 |

Table 3 shows the concentration and PI for the 1-2 and 4-5 μm microbubbles at various time points following size isolation. Both size fractions were stable over two days. Microscopy after two days storage also indicated the persistence of intact microbubbles at their isolated size range over this timeframe. However, results indicated that the microbubbles underwent ripening during longer term storage. For 1-2 μm microbubbles, the concentration decreased from by an order of magnitude, and PI nearly doubled over a period of 28 days. For 4-5 μm microbubbles at less than 1 vol % encapsulated gas, the concentration decreased by more than half, and PI nearly doubled over a period of 14 days. Higher microbubble concentrations provided much greater stability, as seen for the comparison of the 4-5 μm microbubbles in FIG. 8. In general, encapsulated gas fractions were found to be greater than 1 vol % were necessary for good stability, particularly when the vial is intermittently opened to the atmosphere as typically occurs for an in vivo study (data not shown).

When measuring the number-weighted distribution with the Accusizer, the monomodal peak for the 4-5 μm microbubbles changed to a bimodal peak during storage. FIG. 8C shows that 4-5 μm microbubbles degraded into 1-2 μm microbubbles as well as larger microbubbles over the test period. The formation of the larger microbubbles is consistent with Ostwald ripening, in which small microbubbles shrink at the expense of larger ones, as a major factor affecting microbubble stability.

### Example 2

### High Frequency Ultrasonic Imaging Using Isolated Microbubbles

### Materials and Methods

### Materials

NaCl (0.01 M) phosphate buffered saline (PBS) solution was prepared by dissolving salt tablets (Sigma-Aldrich, St. Louis, Mo., USA) in purified water (10 MΩ-cm; Millipore, Billerica, Mass., USA) and adjusting pH to 7.4. PBS was filtered through 0.2-μm pore size polycarbonate filters (V WR, West Chester, Pa., USA). Perfluorobutane (PFB) was obtained from FluoroMed (Round Rock, Tex., USA) at >99 wt % purity. 1,2-distearoyl-sn-glycero-3-phosphocholine (DSPC) was purchased from Avanti Polar Lipids (Alabaster, Ala., USA) and Polyoxyethylene-40 stearate (PEG40S) was obtained from Sigma-Aldrich.

### Microbubble Generation

Microbubbles were prepared as by the techniques described above. Briefly, microbubbles were prepared by sonicating a lipid suspension (DSPC:PEG40S=9:1 molar ratio) in the presence of PFB gas. The microbubble suspension was collected in 60-mL syringes and concentrated into a cake by centrifuging the suspension at 300 relative centrifugal force (RCF) for 5 min using a bucket-rotor centrifuge (Model 5804; Eppendorf, Westbury, N.Y., USA). The microbubble cake was saved and infranatant was discarded. Size populations of 1-2 and 4-5 μm diameter were isolated from the microbubble suspension. A brief description of the size isolation protocol is presented here. All microbubbles were stored at 4° C. Although the size-selected microbubbles are stable upon storage for at least 2 weeks, they were used on the same day on which they were made for experimental consistency.

### Size Isolation of 1-2 μm Bubbles

The initial microbubble sample was collected in 60 mL syringes. Microbubbles greater than 6 μm were removed by centrifuging the initial microbubble suspension at 50 RCF for 1 min. The infranatant containing smaller bubbles was re-suspended to 60 mL with PBS, while the cake containing larger bubbles was discarded. Microbubbles greater than 3 μm diameter were removed by centrifuging the collected infranatant at 290 RCF for 1 min. The infranatant was resuspended to 60 mL with PBS, while the cake was discarded. The collected infrananant was concentrated down to 1 mL using 300 RCF for 5 min. The sample was stored in 2-mL serum vials. The final gas concentration of the suspension was at least 1 vol % to ensure stability.

### Size Isolation of 4-5 μm Microbubbles

Microbubbles less than 4 μm diameter were removed by centrifuging at 120 RCF for 1 min. The cake was collected and reconstituted with PBS, while the infranatant was discarded. This washing step was repeated 5 to 7 times to fully remove smaller bubbles from the sample. Microbubbles greater than 6 μm were removed by centrifuging the resulting suspension at 80 RCF for 1 min. The infranatant containing 4-5 μm microbubbles was collected, while the cake was discarded. The cake was resuspended and centrifuged at 80 RCF for 1 min again to completely remove the larger bubbles. The size-isolation procedure was repeated two to three times to collect a sufficient number of microbubbles. The resulting cakes containing 4-5 μm bubbles were combined and concentrated to 1 mL. The final gas concentration of the 4-5 μm microbubble suspension ranged from 2-5 vol %.

### Size Isolation of 6-8 μm Microbubbles

Microbubbles less than 6 μm diameter were removed by centrifuging at 60 RCF for 1 min. The cake was collected and reconstituted with PBS, while the infranatant was discarded. This washing step was repeated five to seven times to fully remove smaller bubbles from the sample. Microbubbles greater than 8 μm in diameter were removed by centrifuging the resulting suspension at 40 RCF for 1 min. The infranatant containing 6-8 μm microbubbles was collected, while the cake was discarded. The cake was resuspended and centrifuged at 40 RCF for 1 min to completely remove the larger bubbles. The size-isolation procedure was repeated two to three times to produce enough 6-8 μm bubbles for storage. The resulting cakes containing 6-8 μm bubbles were combined and concentrated to 1 mL. The final gas concentration of the 6-8 μm microbubble suspension ranged from 2-10 vol %.

### Polydisperse Microbubbles

Polydisperse lipid-coated microbubbles characterized were produced by the shaking method, which was developed for the commercially available ultrasound contrast agent, Definity. In this procedure, 2 mL of the lipid solution in sterile-filtered PBS (2 mg/mL) was warmed to 60° C. in a sealed 3-mL glass serum vial (Wheaton, Millville, N.J., USA) and briefly bath sonicated to disperse the lipid. The air headspace was exchanged with PFB by attaching the serum vial containing the lipid solution to a three-way valve connected to both the gas canister and vacuum line. The lipid solution was maintained under vacuum to remove the air. The position of the valve was switched to close the vacuum line and immediately flood the headspace with PFB gas. This procedure was repeated five times to ensure complete gas exchange. The pressure in the vial was vented briefly to the atmosphere, and the microbubbles were formed by rapidly shaking the vial for 45 s using a VialMix (Bristol-Myers Squibb, New York, N.Y.). The resulting microbubble suspension was used without further processing.

### Particle Counting and Size Analysis

Microbubble size distributions and concentrations were determined by laser light obscuration and scattering (Accusizer 780A; NICOMP Particle Sizing Systems, Santa Barbara, Calif., USA). Samples (2-μL) of each microbubble suspension were diluted into a 30-mL flask under mild mixing during measurement. Concentrations and size distributions of each sample were measured before injection in triplicate. The Accusizer laser provides uniform illumination across the sample path. Size analysis was confirmed using a Coulter Multisizer III (Beckman Coulter, Opa Locka, Fla., USA). A 5-4 sample of microbubble suspension was diluted into a 60-mL flask and stirred continuously to prevent flotation-induced error. A 30-μm aperture (size range of 0.6-18 μm) was used for the measurements.

### Animal Injections

All animal experiments were approved by the Columbia University Institutional Animal Care and Use Committee. Contrast persistence studies were performed in female CD-1 mice 4 to 6 weeks of age (Charles River Laboratories, Wilmington, Mass., USA). Mice were anesthetized using 1% to 2% isofluorane and placed on a mouse handling table, and the heart rate, respiratory rate and temperature were monitored using a TMH-150 physiological monitoring unit (Visualsonics, Toronto, Ontario, Canada). Mice were kept under anesthesia for the duration of the experiment. After the mouse was completely anesthetized, the tail vein was catheterized using a modified 27-gauge, one half-inch butterfly catheter (Terumo Medical Corporation, Somerset, N.J., USA). Prior to catheterization, the tubing was removed from the butterfly catheters and replaced with smaller 27-gauge Tygon tubing (0.015-inch inner-diameter, Cole-Farmer, Vernon Hills, Ill., USA). The mouse was then shaved in the kidney region. A Vevo 770 small animal ultrasound imaging scanner with a 40-MHz imaging transducer was placed over the kidney region and coupled using Aquasonic-100 ultrasound transmission gel (Parker Laboratories, Fairfield, N.J., USA). A bolus injection of 100 μL of microbubble suspension (concentrations range from 1×10⁶ to 1×10⁸ MB/bolus) was injected while imaging continuously at 16 frames per s. At the focal length (6 mm), the peak negative pressure is 3.49 MPa with an acoustic power of 0.017 mW and a mechanical index of 0.57. In the current work, the transmit power setting was set to 79%, corresponding to 1 dB attenuation of the signal.

These acoustic parameters were reported by the manufacturer (Visualsonics) from measurements made in water and adjusted with a derating factor of 0.3 dB cm⁻¹ MHz⁻¹. Mice were injected with size-selected microbubble populations of 1-2 μm, 4-5 μm or 6-8 μm at specific number concentrations ranging between 1×10⁶ and 1×10⁸ MB/bolus (n=4-7 per group). Respiratory gating was used to synchronize data acquisition with the mouse respiratory cycle to reduce motion artifact during image analysis. Respiratory gating lowered the effective acquisition rate to 2 frames per s. Ultrasound imaging was performed from 0 to 5 min. For longer circulating bubbles, imaging was performed from 0 to 20 min following injection of the microbubble solution. Mice were given up to three injections per imaging session (20 min was given between start points of the injections) and then removed from anesthesia. After the mouse regained consciousness, it was returned to its cage.

### Data Analysis

Image analysis of the contrast enhancement was performed using the mean grayscale video intensity determined by integrating the pixel intensity values over a specific region-of-interest (ROI) in every acquired frame. The ROI was selected in the upper portion of the kidney to minimize effects of signal attenuation and shadowing. The log-compressed grayscale video intensity values were linearized using the Vevo770 application software (Visualsonics). This method was performed by characterization of input/output relationship of the 40 MHz transducer to calculate the input voltage from the grayscale pixel values. The relationship between the grayscale value and input voltage is given as:

y=m ln(x)+b

where y is the grayscale value and x is the input voltage. The slope m and offset b were determined to be 369.39 and 371.86, respectively. The details regarding the validation of this method can be found in the VSI White paper (available through Visualsonics). The linearized data were baseline adjusted and plotted using Prism 5 graphing software (Graphpad Software, La Jolla, Calif., USA).

The amplitude, persistence half-life, decay rate and total integrated signal enhancement (area under the curve; AUC) of the contrast signal was measured by fitting the data to a drug persistence model, in which agent is absorbed at a first-order rate into the intravascular compartment, mixes immediately and then decays at a first-order rate. See the section below entitled “Theories and Mathematics for Data Analysis” for details. TIC data were fit to the pharmacokinetic model using a least-squares regression algorithm. All statistical analyses were performed using Prism 5 software.

### Theories and Mathematics for Data Analysis

A. Pharmacokinetic Model for Contrast Persistence:

The pharmacokinetic model used to fit contrast time-intensity curves was given by:

$$\frac{C}{{D}_{o}} = \frac{k 1}{( k 2 - k 1 ) ( {e}^{- k1 * t} {e}^{- k 2 * t} )} \qquad ( 4 )$$

where C is the relative amount of measured contrast enhancement, Dₒ is the initial amount of contrast, k₁ is a pseudo-first-order rate constant describing the influx of contrast into the system and k₂ is a pseudo-first-order rate constant describing the elimination of contrast from the system.

B. Estimation of the Extinction Cross-Section:

The extinction cross section was given by:

σₑ=σₛ+σₐ (5)

The extinction cross-section (σₑ) is the total energy of the ultra-sound beam lost when passing through the microbubble-containing medium and is defined by the sum of the scattering cross-section (σₛ) and absorption cross-section (σₐ) of the microbubbles. The scattering and absorption cross sections of the bubble are given as:

$$\begin{matrix}{σ}_{s} = \frac{4 π {r}^{2}}{{( \frac{{f}_{r}}{f} - 1 )}^{2} + {δ}^{2}} & ( 6 ) \\ {σ}_{a} = {σ}_{s} ( \frac{δ}{{δ}_{r}} - 1 ) & ( 7 )\end{matrix}$$

where r is the microbubble radius, fᵣ is the resonance frequency of the microbubble, f is the driving frequency of the acoustic pulse, δ is the overall damping coefficient, and δ_{R} is the damping coefficient for re-radiation. At 40 MHz the ultrasound wavelength begins to approach the size of the microbubbles tested in this Example. However, the theory does provide some physical insight into the experimental results.

The resonance frequency fᵣ is given as:

$${f}_{r} = {{f}_{RA} ( b ξ )}^{\frac{1}{2}} + \frac{1}{2 π} {( \frac{{S}_{shell}}{m} )}^{\frac{1}{2}} \qquad ( 8 )$$

where f_{RA} from eqn (8) is the natural frequency of the free oscillation, ξ is a substitution variable, b is a dimensionless stiffness factor, Sₛₕₑₗₗ is the shell stiffness and m is the effective mass. Total damping (δ) is a sum of the re-radiation, (δᵣ) shear viscous losses in the surrounding medium (δᵥ), thermal transport between the gas and liquid (δ_{T}) and frictional damping (δ_{F}).

δ=δᵣ+δ_{υ}+δ_{T}+δ_{F} (9)

C. Estimation of the Bubble Resonance Frequency:

The natural frequency of the free oscillation given as:

$${f}_{RA} = \frac{1}{2 π r} {( \frac{3 γ {P}_{0}}{ρ} )}^{\frac{1}{2}} \qquad ( 10 )$$

where P₀ is the ambient pressure, ρ is the density of the liquid, and γ is the ratio of constant-volume specific heat to constant-pressure specific heat. ξ from eqn (8) is a substitution factor defined as:

$$ξ = 1 + \frac{2 β}{{P}_{0} r} [ 1 - \frac{1}{3 γ b} ] \qquad ( 11 )$$

Where β is the surface tension and b is defined by:

$$b = {{( 1 + {B ( ω , r )}^{2} )}^{- 1} [ 1 + \frac{3 ( γ - 1 )}{X} ( \frac{sinh X + sin X}{cosh X - cos X} ) ]}^{- 1} \qquad ( 12 )$$

where B (ω,r) and X are defined by eqns (16) and (17) below, respectively.

D. Estimation of the Damping Coefficients:

Re-radiation (δᵣ) is defined as:

δᵣ=kr (13)

where k is the wave number. Viscous damping (δ_{υ}) is defined as:

$${δ}_{v} = 4 \frac{η}{ρω {r}^{2}} \qquad ( 14 )$$

where η is the viscosity of the liquid, ρ is the density of the liquid and ω is the applied angular frequency. Thermal damping (δ_{T}) is defined by:

$${δ}_{T} = B ( ω , r ) \frac{{ω}_{T}^{2}}{{ω}^{2}} \qquad ( 15 )$$

where B (ω,r) is defined as:

$$B ( ω , r ) = 3 ( γ - 1 ) [ \frac{X ( sinh X + sin X ) - 2 ( cosh X - cos X )}{{X}^{2} ( cosh X - cos X ) + 3 ( γ - 1 ) X ( sinh X + sin X )} ] \qquad ( 16 )$$

and X is defined by:

$$X = {r ( \frac{2 {ωρ}_{g} {C}_{pg}}{{K}_{g}} )}^{\frac{1}{2}} \qquad ( 17 )$$

where Cₚ is the constant-pressure specific heat, K_{g} is the thermal conductivity of the gas, and ρ_{g} is the gas density defined by:

$${ρ}_{g} = {ρ}_{g} A [ 1 + \frac{2 β}{{P}_{0} r} ] \qquad ( 18 )$$

where ρ_{g}A is the gas density at ambient pressure, β is the surface tension and P₀ is the ambient pressure. The angular resonance frequency in eqn (15) is defined by:

$${ω}_{r}^{2} = {ω}_{rg}^{2} + \frac{{S}_{shell}}{m} \qquad ( 19 )$$

where w_{rg}² is the angular resonance frequency of an ideal gas bubble defined as:

$${ω}_{rg}^{2} = \frac{{S}_{α}}{m} b β \qquad ( 20 )$$

where Sₐ is the adiabatic stiffness of the gas defined as:

Sₐ=12πγP₀r (21)

where γ is ratio of constant-volume specific heat to constant-pressure specific heat. Frictional damping (δ_{F}) is defined by:

$${δ}_{F} = \frac{{S}_{f}}{ω m} \qquad ( 22 )$$

where S_{f} is the shell friction coefficient.

The numerical values for the parameters defined in sections B-D are given in Table 4.

TABLE 4
Parameters used to estimate scattering and adsorption cross-sections.
Parameters

| | |
|---|---|
| f - Frequency [MHz] | 40 |
| γ = Cᵥ/Cₚ | 1.3 |
| Speed of sound [m/s] | 1497 |
| P₀ - Ambient pressure [Pa] | 101325 |
| ρ - Density of water [kg/m³] | 1000 |
| C_{pg} - Heat capacity [J/K] | 406.51 |
| ρ_{g}A - Ambient density of PFB [kg/m³] (Ideal) | 9.7 |
| K_{g} - Thermal conductivity of PFB [W/(m K)] | 0.0143 |
| S_{f} - Shell friction damping coefficient [kg/s] × 10⁻⁸ | 1 |
| Sₛₕₑₗₗ - Shell stiffness [N/m] | 27 |
| η - Viscosity of water [Pa s] × 10⁻⁴ | 6.92 |
| Sₐ - Gas stiffness [N/m] | 24.829 |
| σ - Surface tension [mN/m] | 0-72 |

### Results

### Microbubble Size Distribution

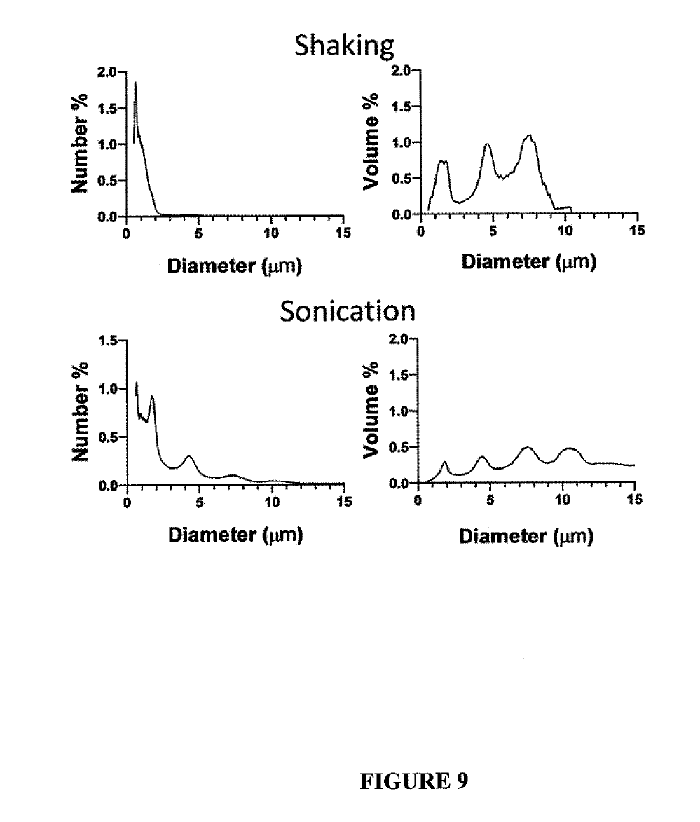

FIG. 9 shows the number- and volume-weighted size distributions from freshly made microbubble suspensions formulated by vial shaking and probe sonication. (Microbubbles were formulated using a Vialmix shaker for small volumes of lipid solution (2 mL) and probe sonication for large volumes of lipid solution (120 mL). All lipid solutions (2 mg-lipid/mL) consisted of DSPC and PEG40S at a 9:1 molar ratio.) Both methods resulted in microbubbles with multimodal size distributions, with diameters ranging from 0.5 μm to 10 μm in diameter. The number-weighted size distributions showed that most (>95%) of the microbubbles are smaller than 2 μm diameter. The volume-weighted distributions showed that subpopulations of microbubbles existed at distinct size ranges (specifically at 1-2 μm, 4-5 μm and 6-8 μm diameter), as indicated by the peaks.

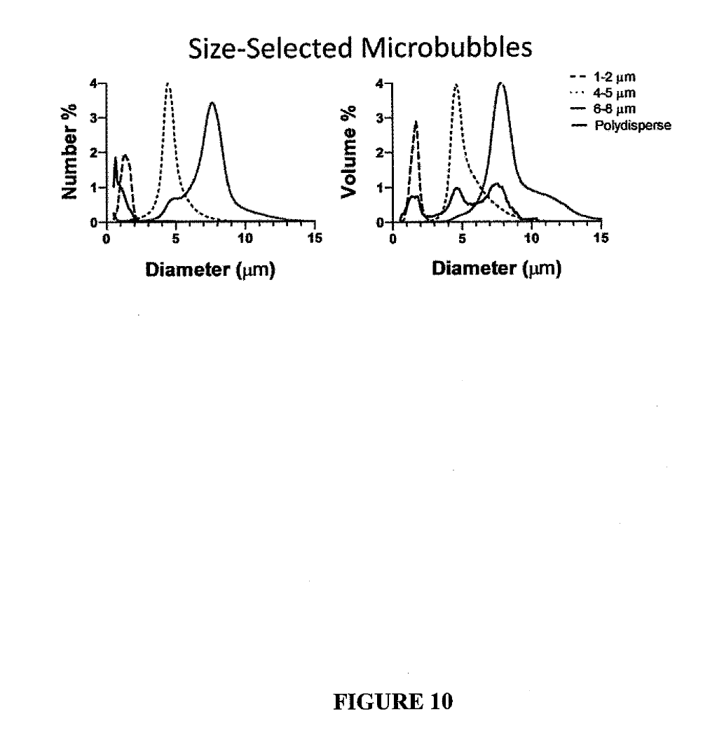

The size-isolation technique as described above was used to separate microbubbles at 1-2 μm, 4-5 μm and 6-8 μm size ranges in high concentrations (10⁷ to 10⁹ MB/mL). FIG. 10 shows representative number- and volume-weighted size distributions for the size-selected microbubble populations used in this example. (The microbubble populations for the individual 1-2 μM, 4-5 μm and 6-8 μm, samples are shown as number-weighted and volume-weighted size distributions. The polydisperse sample is from a freshly made microbubble suspension generated using a shaker.) Differential centrifugation allowed rapid and efficient isolation of size-selected microbubbles with monomodal distributions and relatively low polydispersity in comparison to the original suspension.

### High Frequency In Vivo Imaging

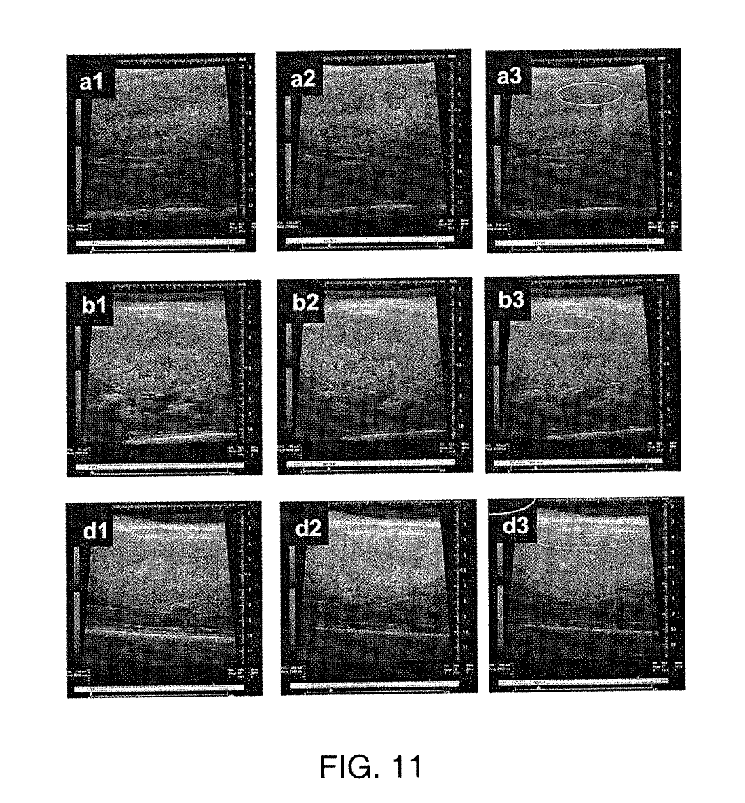

All mice were given 100-4 bolus injections ranging from 10⁶ to 10⁸ microbubbles total. Imaging was performed above the left kidney of the mouse using a 40-MHz ultrasound transducer to record ultrasound videos in fundamental mode. FIG. 11 shows typical grayscale images of the mouse kidney before and after a bolus injection of 5×10⁷ microbubbles for polydisperse and size-selected microbubbles. The images were taken at the peak amplitude of the signal, typically 30 to 60 s after the bolus injection was given. Microbubble suspensions of 100 μL containing 5×10⁷ MB of (11A) polydisperse, (11B) 1-2 μm, (11C) 4-5 μm or (11D) 6-8 μm diameter bubbles were injected intravenously into anesthetized mice while continuously imaging the kidney using a 40-MHz ultrasound probe. Gray-scale images are shown before the bolus is injected (A1-D1) and at the peak signal intensity (A2-D2), typically 30 to 60 s after the bolus was delivered. Contrast detection software (Visualsonics), which colors the positive contrast-enhanced pixels in green (A3-D3), was used to highlight the presence of microbubbles. Areas outlined in white are regions-of-interest (ROI) selected for time-intensity curve (TIC) analysis.)

The 1-2 μm bubble population showed little increase in the grayscale video intensity after the bolus injection (FIG. 11C2), compared with a typical polydisperse sample (FIG. 11A2). Contrast detection software indicated sparse signal increase throughout the kidney except in large blood vessels (>100 μm in diameter). However, attenuation of the ultrasound signal was observed in the lower portion of the kidney and below.

The 4-5 μm bubble population showed a noticeable increase in the brightness and speckling throughout the kidney after the bolus injection (FIG. 11C2), compared with background images prior to the injection. Contrast detection software similarly indicated a higher level of contrast enhancement (indicated by the area and intensity of green color) following injections of the 4-5 μm bubbles, compared with injections of the 1-2 μm bubbles.

The 6-8 μm bubble population showed the largest increase in brightness and speckling following the bolus injection (FIG. 11D2). The contrast detection software also indicated higher levels of contrast enhancement from the 6-8 μm bubbles compared with both 4-5 μm and 1-2 μm bubbles. The 6-8 μm microbubbles consistently gave the most intense and longest lasting positive contrast enhancement.

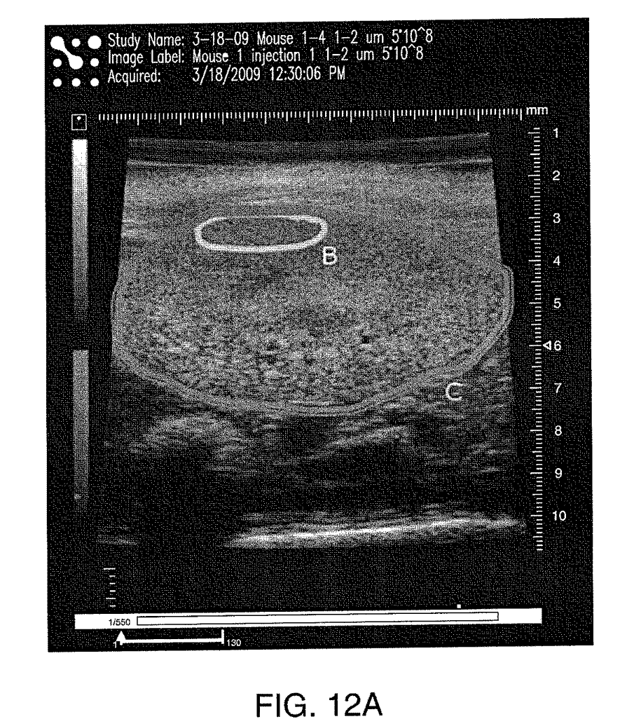

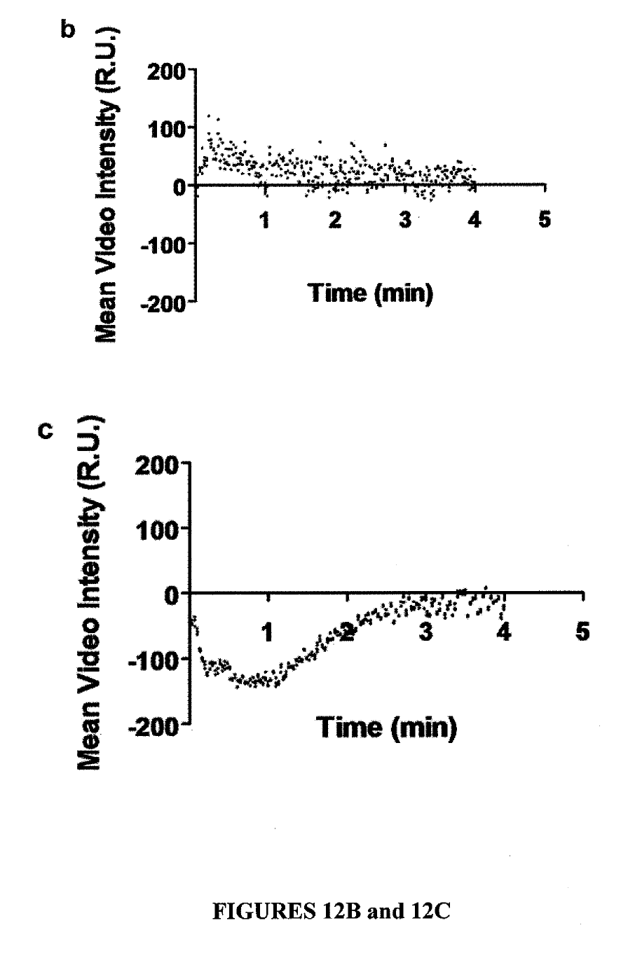

The levels of contrast enhancement and persistence of the contrast agent in the blood stream were analyzed from the time-intensity curves (TIC) for each of the size-selected microbubble populations. Typically, the time-intensity curves were generated by integrating the image pixel intensities over an ROI in the upper portion of the kidney (FIGS. 11 and 12). FIG. 12 depict attenuation of the ultrasound signal from 1-2 μm bubbles. The change in video intensity caused by the 1-2 μm bubbles was evaluated over two regions-of-interest (ROI). Typically, a smaller ROI in the upper portion of the kidney (yellow) was used to avoid effects of attenuation and shadowing. The larger ROI (green) encompassed areas deeper within the tissue, which were prone to shadowing and attenuation by microbubbles (12A). The relative acoustic backscatter of the ultrasound signal from the 1-2 μm bubbles showed little signal enhancement above noise in the smaller ROI ((12B). The 1-2 μm bubbles showed strong attenuation of the signal, as determined by whole kidney as the ROI (12C).

The upper portion was selected to minimize effects of microbubble-induced attenuation and shadowing, particularly at higher microbubble concentrations. Interestingly, the 1-2 μm bubble population did not show a measurable signal increase above noise when evaluating the mean video intensity in the upper portion of the kidney (FIG. 12B). A small amount of signal enhancement was observed qualitatively but could not be accurately quantified due to a very low signal-to-noise ratio. However, the presence of microbubbles in circulation was detectable by attenuation of the signal (FIG. 12C). By selecting an ROI that encompassed the entire kidney, a decrease in the mean video intensity was observed (FIG. 12C), and the circulation persistence of the 1-2 μm bubbles could be measured.

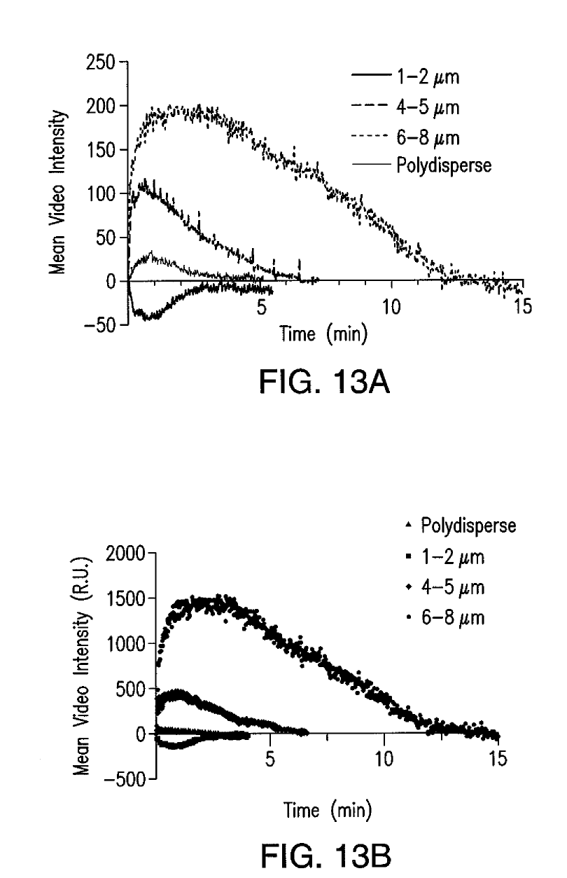

FIG. 13 shows typical TIC generated from individual bolus injections of 5×10⁷ MB for each size-selected population. (FIG. 13A shows the log-compressed mean video intensity and FIG. 13B shows the linearized mean video intensity. Representative time-intensity curves (TIC) are shown for each size-selected population after a 100 μL bolus injection of 5×10⁷ MB. All data was offset corrected using background images as a reference.) Injections of 4-5 μm and 6-8 μm bubbles significantly enhanced the video intensity compared with both 1-2 μm bubbles and polydisperse samples injected at the same number concentration. Larger 4-5 μm and 6-8 μm microbubbles were observed to persist longer in circulation than 1-2 μm and polydisperse bubbles.

### Pharmacokinetic Model

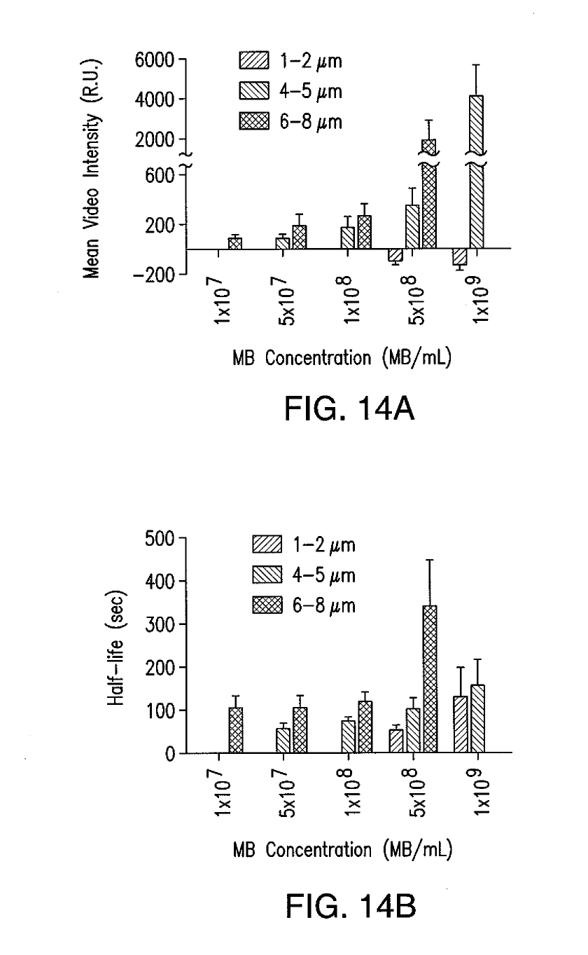

A pharmacokinetic model was used further analyze the TIC data to determine the signal amplitude and contrast persistence half-life over a range of microbubble concentrations (10⁶ to 10⁸ MB/bolus) for each of the size-selected microbubble populations. FIG. 14 depicts signal amplitude and half-life of size-selected microbubbles. The signal amplitude and total contrast persistence was measured from the time-intensity curves (TIC) over a range of microbubble concentrations. The signal amplitude was determined by the maximum signal intensity from the TIC for each microbubble sample (14A). For 1-2 μm bubbles which attenuated the signal, the minimum signal intensity value was used. The half-life of the signal was determined from the TIC at the time the signal intensity decayed to half of the maximum amplitude (14B).

The model assumes a first-order rate of bolus spreading following injection, uniform distribution of the microbubbles in circulation and a first-order rate of elimination of the microbubbles from circulation. While these assumptions may not fully describe the kinetics of microbubble influx and elimination, the model fits the data well (typical R²>0.90) and allows parameters such as the maximum signal intensity and half-life to be quantified.

Attenuation of the signal from 1-2 μm bubbles was only detectable above 5×10⁷ MB/bolus. The change in signal intensity was greater at 1×10⁸ MB/bolus (−128±44 relative units [R.U.]) compared with 5×10⁷ MB/bolus (94±32 R.U.), but the change was not statistically different (p=0.23), as determined by a Students' t-test. The half-life of the 1-2 μm bubbles significantly increased, however, from 51±22 s to 115±24 s (p<0.05).

The 4-5 μm bubble population showed a positive signal enhancement for concentrations ranging from 5×10⁶ to 1×10⁸ MB/bolus. Both signal amplitude and persistence time increased with microbubble concentration. The signal amplitude increased from 82±33 R.U. at the lowest concentration of 5×10⁶ MB/bolus to 4081±1575 R.U. at the highest concentration of 1×10⁸ MB/bolus. Similarly, the persistence half-life increased from 66±9 s to 117±21 s for injections of 5×10⁶ and 1×10⁸ MB/bolus. The signal amplitude and contrast persistence were significantly different for each concentration (p<0.05).

Similarly, the 6-8 μm bubble population showed a positive signal enhancement for concentrations ranging 1×10⁶ to 5×10⁷ MB/bolus. As with the 4-5 μm bubbles, both signal amplitude and persistence time increased with microbubble concentration. The signal amplitude increased from 82±31 R.U. at the lowest concentration of 1×10⁶ MB/bolus to 1886±993 R.U. at the highest concentration of 5×10⁷ MB/bolus. The half-life increased from 119±31 s to 350±84 s for injections of 1×10⁶ and 5×10⁷ MB/bolus. Interestingly, one-way analysis of variance (ANOVA) determined that there was no significant change in the persistence half-life for concentrations ranging from 1×10⁶ to 1×10⁷ MB/bolus (p=0.62). However, bolus injections of 5×10⁷ bubbles showed a dramatic increase (threefold) in the persistence half-life compared with the lower concentrations (p<0.01).

The signal intensities and contrast persistence times were compared between the individual size populations as well. At the highest comparable concentration of 5×10⁷

MB/bolus, the 6-8 μm bubbles demonstrated a 5.4-fold increase in the signal intensity compared with the 4-5 μm bubbles (p<0.01). At 1×10⁷ and 5×10⁶ MB/bolus, there was no statistical difference in the signal intensities between the 4-5 μm and 6-8 μm bubbles (p<0.23 and 0.10, respectively). Concentrations greater than 5×10⁷ MB/bolus for the 6-8 μm bubbles were difficult to achieve owing to the small population of the microbubbles in the initial suspension.

The signal half-life from the 6-8 μm bubbles was significantly longer than the 4-5 μm and 1-2 μm populations for every comparable concentration (p<0.01). At the highest comparable concentration of 5×10⁷ MB/bolus, the half-life of the 6-8 μm bubbles was 350±84 s, which was fourfold longer than the 4-5 μm bubbles and sevenfold longer than the 1-2 μm bubbles. The signal from the 4-5 μm bubbles did not have a significantly longer half-life than the 1-2 μm bubbles at 5×10⁸ MB/bolus (p=0.08), or at 1×10⁸ MB/bolus (p=0.14).

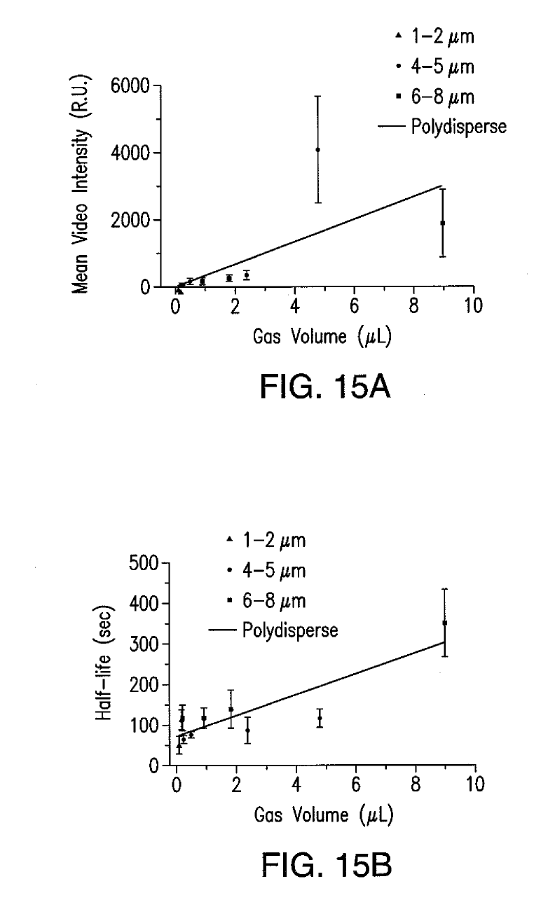

The effect of the total gas volume injected on the signal amplitude and half-life was explored. Total injected gas volume was determined by the size distribution and the total number of bubbles (FIG. 15, which depicts signal amplitude and half-life of size-selected microbubbles as a function of total gas volume. The signal amplitude values (15A) and half-life values (15B) are the same as shown in FIG. 14). The total gas volume injected for the highest concentration of 1-2 μm bubbles (0.17 μL at 10⁸ MB/bolus) was moderately less than the volume injected for the lowest concentrations of 4-5 μm (0.23 μL at 5×10⁷ MB/bolus) and 6-8 μm (0.18 μL μm³ at 10⁶ MB/bolus) but of the same order of magnitude. At these microbubble concentrations, the signal from the 1-2 μm bubbles mainly attenuated without producing a measurable increase in the video signal, whereas 4-5 μm and 6-8 μm bubbles demonstrated a clear signal enhancement (FIG. 15A). The relationship between gas volume and signal amplitude was nonlinear, particularly for 4-5 μm bubbles, indicating the onset of multiple scattering events at the higher microbubble concentrations.

At the equivalent gas volume (˜0.2 μL), the half-life of 1-2, 4-5 and 6-8 μm bubbles was 135±65, 56±34 and 106±27 s, respectively. These values are remarkably similar, although the nearly twofold longer half-life for 1-2 μm and 6-8 μm vs 4-5 μm bubbles was statistically significant (p<0.01). The shorter half-life for 4-5 μm bubbles is likely due to an underestimation from the method of analysis, as discussed below. The relationship between gas volume and signal half-life had a moderate linear correlation (R²=0.75).

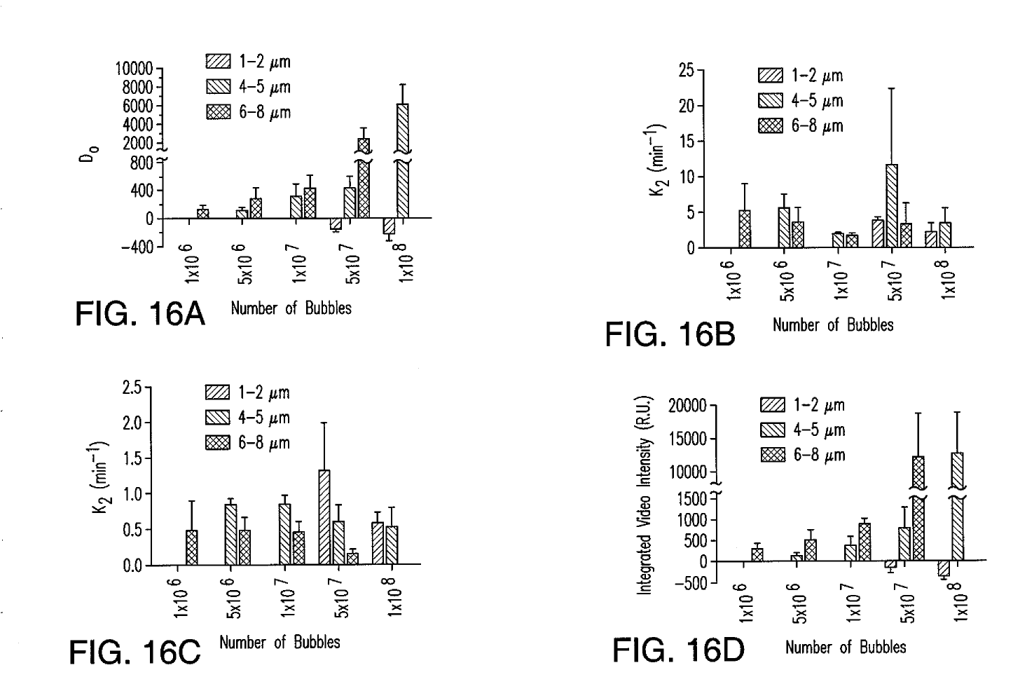

The parameters Dₒ, k₁, k₂ and the AUC are shown in FIG. 16. In FIG. 16, each TIC was fit to a model describing the influx and decay of contrast agent in the kidney [eqn (4) below]. The parameters were determined by fitting the data to the equation using least a squares regression analysis.

The Dₒ parameter (FIG. 16A) is directly related to the maximum signal intensity. The Dₒ values estimated by the model fit showed an identical trend to the maximum signal amplitudes shown in FIG. 14A.

The k₁ parameter (FIG. 16B) is a measure of the influx rate of contrast agent into the kidney, which depends on the injection and bolus spreading. No trend was observed for any of the size-selected microbubble populations with increasing concentration. For example, the effect of increasing microbubble concentration for 4-5 and 6-8 μm bubbles was analyzed using one-way ANOVA. No statistical difference in the mean k₁ values with increasing microbubble concentration was found (p=0.21 and p=0.32, respectively).

The k₂ parameter (FIG. 16C) is a measure of the decay of contrast agent in the circulation. The mean k₂ values were significantly lower for the 6-8 μm bubble compared with the 4-5 μm and 1-2 μm bubbles at equivalent concentrations (p<0.01). No statistical difference was observed between mean k₂ values for 4-5 μm and 1-2 pin bubbles at any comparable concentration.

The AUC parameter is a measure of the total integrated signal enhancement and is the most relevant measure of the capability of the microbubbles to produce contrast. The AUC was determined to be significantly higher for the 6-8 μm bubbles than for the 4-5 μm bubbles at equivalent concentrations (p<0.01). For example, at the highest concentration of 6-8 μm bubbles injected, the mean AUC was 16-fold higher than the mean AUC for the 4-5 μm bubbles at the same concentration. The AUC of 1-2 μm bubbles are shown as negative values due to the attenuation of the signal.

Based on the above results, in vivo imaging of the size-selected microbubble populations illustrated that larger microbubbles (>4 μm diameter) enhanced the video signal more effectively at 40 MHz than smaller bubbles in fundamental imaging mode. Small bubbles, between 1-2 in size, minimally enhanced the ultrasound signal in the mouse kidney, but they strongly attenuated (FIGS. 11-13). This result is consistent with theoretical predictions, in which smaller microbubbles are expected to absorb HFU to a greater degree than they scatter at the fundamental frequency.

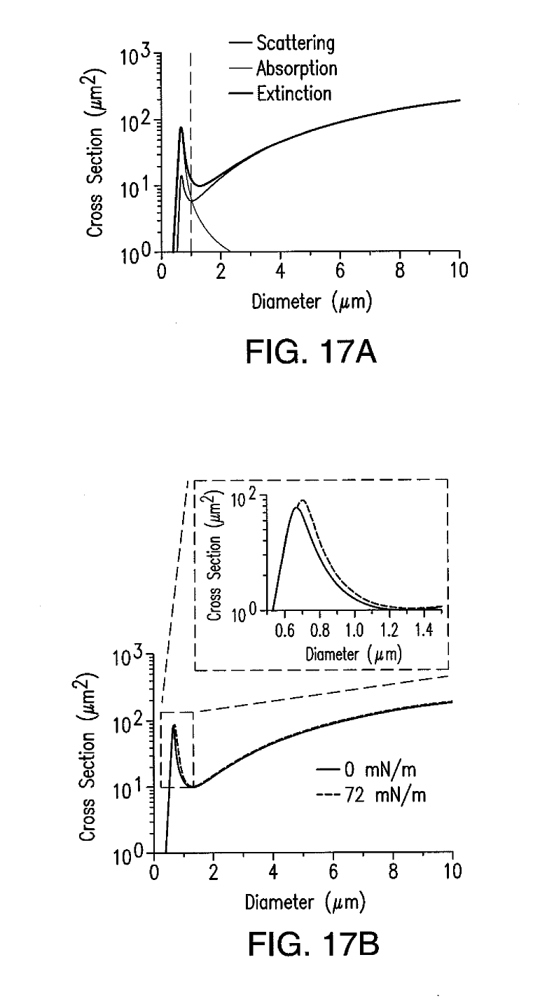

To gain physical insight into the in vivo imaging data, the relative contributions of the scattering cross section (σₛ) and absorption cross section (σₐ) to the extinction (attenuation) cross-section (σₑ) at 40 MHz were calculated as a function of microbubble size. The estimates are based on Rayleigh-Plesset theory with an additional damping coefficient due to frictional loss. The equations and parameters used in the calculations are given in the Appendix. The results are shown in FIG. 17. (FIG. 17A shows extinction cross-section as a function of diameter. The extinction cross section, absorption cross section and scattering cross section were calculated using equations derived by Medwin with an additional damping coefficient due to frictional loss; FIG. 17B shows the effect of surface tension on extinction cross-section. The extinction-cross-section as a function of diameter was plotted for low (0 mN/m) and high (72 mN/m) surface tensions to determine the effect of rapidly changing surface tension on the microbubble shell in an ultrasound field. Grey area represents surface tensions between 0 and 72 mN/m.) Larger microbubbles are predicted to produce more scattering and less absorption. The amount of scattering predicted for a 4-5 μm bubble is fivefold higher than for a 1-2 μm bubble, while that for a 6-8 μm bubble is 10-fold higher than for a 1-2 μm bubble. At microbubble diameters between 1 and 2 μm, the model predicts energy loss primarily owing to absorption of the ultrasound signal. Loss of energy at the fundamental due to absorption effectively reduces the scattering-to-attenuation ratio (STAR), a value that was developed to describe the efficiency of microbubble contrast agents to enhance visualization in tissue. For small microbubbles close to 1 μm in diameter, it is estimated from FIG. 17A that 50% of the attenuation is due to absorption (FIG. 17).

Note that resonant microbubbles are more efficient scatterers when volume-fraction matched. The 1-2 μm bubbles are nearest the resonant peak at 40 MHz (FIG. 17) but they are not resonant. The result remains, however, that larger microbubbles provide greater contrast enhancement when imaging at depth, which is dominated by the STAR effect.

Other experimental factors can explain to the inability of the 1-2 μm bubbles to measurably enhance the video intensity. It is possible that an increase in the video signal can become discernable from noise by increasing the applied ultrasound amplitude or imaging alternate tissue. Interestingly, it was observed that 1-2 μm bubbles showed a significant increase in video intensity in the large blood vessels (>100 μm in diameter) of the kidney. These large blood vessels were characterized by dark circles within the kidney that became significantly brighter following the bolus injection of 1-2 μm bubbles.

It is unlikely that the lower enhancement (shadowing) observed for smaller microbubbles is simply a consequence of larger numbers reaching the kidney region. At each matched number concentration (1×10⁶ to 1×10⁸ MB/mL), the larger microbubbles consistently showed greater contrast enhancement and longer circulation persistence (FIG. 14). No rise and then drop in enhancement was observed with increasing dose for any of the sizes. Furthermore, the results are in line with theory, which predicts a lower STAR value for the smaller microbubbles (FIG. 17).

The effect of surface tension on the model predictions was also investigated. The extinction cross section of microbubbles at 40 MHz was calculated as a function of microbubble radius for negligible (0 mN/m) and high (72 mN/m) surface tension. FIG. 17B shows that surface tension had little effect on attenuation of the acoustic signal. This analysis does not preclude other effects of the lipid shell, such as surface dilatational viscoelasticity.

The absolute change in contrast increased with microbubble concentration for all sizes. A concentration-dependent increase in signal amplitude was previously demonstrated for nonlinear HFU using polydisperse Definity microbubbles. An increase in backscatter has also been observed with increasing microbubble concentration at lower ultrasound frequencies. However, while increasing concentration enhanced contrast in the upper region of the kidney, it also enhanced attenuation and shadowing in the lower region of the kidney. Therefore, microbubble concentration must be carefully titrated to provide the desired enhancement at a specific imaging depth.

The lowest total gas volumes injected for the 4-5 μm and 6-8 μm bubbles were nearly the same as the highest concentration injected for the 1-2 μm bubbles (˜0.2 μL). Both 4-5 μm and 6-8 μm bubbles showed a positive contrast enhancement, whereas the 1-2 μm mainly attenuated the signal, indicating that the amount of contrast enhancement is not determined by the volume of the gas that is injected but rather how the gas is distributed within the microbubbles.

The time-intensity curves (TIC) of the size-selected microbubbles indicate that larger microbubbles unexpectedly persist longer in the blood stream, especially at high concentrations. Clearance of the microbubbles from circulation has been attributed to (1) filtering of microbubbles by the lung and spleen, (2) dissolution of the microbubble gas core and (3) removal by macrophages in the lungs liver and spleen. Filtering of the microbubbles and macrophage clearance predict that larger microbubbles would be removed more rapidly. Based on the longer contrast persistence of larger bubbles, it can be inferred that dissolution of the gas core is the dominant mechanism of contrast decay. It is believed that larger microbubbles can deform to traverse the capillary beds without becoming lodged and occluding flow. Microbubble deformation has previously been observed both in vitro and in vivo.

At the only comparable gas volume concentration (˜0.2 μL), the 1-2, 4-5 and 6-8 μm diameter microbubbles had similar persistence half-lives. This result is consistent with gas dissolution as the primary mechanism of contrast decay. Contrast elimination was further explored by fitting the TIC data to a pharmacokinetic model and evaluating the k₂ parameter (FIG. 16C). The k₂ parameter was significantly lower for the 6-8 μm bubbles than 1-2 μm or 4-5 μm bubbles. However, no significant differences were observed between 1-2 μm and 4-5 μm bubbles at comparable number concentrations. Again, this is consistent with dissolution as the main mechanism for contrast decay. It is likely that the persistence of 4-5 μm bubbles was underestimated by this analysis, since they dissolved into 1-2 μm bubbles, with low STAR values, and thus became difficult to detect by enhancement of the upper region of the kidney (compared with attenuation of the entire kidney, as measured for 1-2 μm bubbles). The 6-8 μm bubbles, on the other hand, dissolved into signal-enhancing 4-5 μm bubbles before they dissolved into signal-attenuating 1-2 μm bubbles.

Using the technique of differential centrifugation, microbubbles of distinct size populations were isolated and their acoustic imaging impact in vivo was determined The acoustic and pharmacokinetic behavior of 1-2, 4-5 and 6-8 μm diameter microbubbles were measured in the mouse kidney using 40-MHz fundamental-mode imaging. Interestingly, small 1-2 μm diameter microbubbles did not produce a noticeable contrast enhancement compared with tissue at any concentration. In fact, 1-2 μm bubbles mainly attenuated the ultrasound signal of the mouse kidney. This observation was in agreement with predictions of the scattering and absorption cross-sections that predicted 1-2 μm bubbles would have a low STAR at 40 MHz. Larger microbubbles (>4 μm), on the other hand, were highly echogenic and persisted longer in the mouse circulation. The largest microbubbles (6-8 μm) produced a detectable signal even at very low microbubble concentrations (10⁶ MB/bolus). The 6-8 μm bubbles had the longest persistence, lasting more than 15 min in circulation at the highest concentrations (5×10⁷ MB/bolus).

It is important to note that the size distributions of the microbubbles are likely to evolve immediately following the bolus injection, which has not been addressed in this Example. For example, changes in temperature, pressure and ambient gas concentrations can impact microbubble size when delivered in vivo. The size change of the microbubbles following the bolus injections is a complex issue. While an increase in temperature from 25 to 37° C. can cause a slight increase in volume of the microbubbles (a maximum increase in volume by a factor of 1.04 assuming the ideal gas law and no change in internal pressure), other factors such as gas influx into the core and microbubble dissolution can have a more dramatic impact on the microbubble size following injection. Regardless, the larger microbubbles circulated longer with no adverse effects observed in the mice following the imaging protocol, indicating they are not growing upon injection and lodging in capillaries.

Commercially available ultrasound contrast agents, such as Definity (Lantheus Medical Imaging) and SonoVue (Bracco), typically comprise a majority of microbubbles below 2 μm diameter. These results indicate that a very small percentage of the microbubbles (those larger than 2 μm diameter) in these formulations are contributing most to the contrast enhancement. The more numerous, smaller microbubbles actually reduce contrast. Therefore, an optimized contrast formulation for fundamental mode FUS imaging would comprise larger microbubbles.

The foregoing merely illustrates the principles of the disclosed subject matter. Various modifications and alterations to the described embodiments will be apparent to those skilled in the art in view of the teachings herein. It will be appreciated that those skilled in the art will be able to devise numerous modifications which, although not explicitly described herein, embody the principles of the disclosed subject matter and are thus within its spirit and scope.

What is claimed is:

1. A method for isolating target microbubbles having a predetermined size range from polydisperse microbubbles, comprising:
applying a first centrifugal field having a first field strength to a suspension comprising the polydisperse microbubbles for a first duration of time, thereby forming a first infranatant comprising at least a portion of target microbubbles and a first supernatant cake comprising microbubbles having a greater size than the target microbubbles;
removing the first supernatant cake;
applying a second centrifugal field having a second field strength to the first infranatant for a second duration of time, the second field strength being greater than the first field strength, thereby forming a second supernatant cake comprising at least a portion of the target microbubbles and second infranatant comprising microbubbles having a smaller size than the target microbubbles; and
isolating the second supernatant cake.

2. A method for isolating target microbubbles having a predetermined size range from polydisperse microbubbles, comprising:
applying a first centrifugal field having a first field strength to a suspension comprising the polydisperse microbubbles for a first duration of time, thereby forming a first infranatant comprising at least a portion of target microbubbles and a first supernatant cake comprising microbubbles having a greater size than the target microbubbles;
removing the first supernatant cake;
applying a second centrifugal field having a second field strength to the first infranatant for a second duration of time, the second field strength being greater than the first field strength, thereby forming a second infranatant comprising at least a portion of target microbubbles and a second supernatant cake comprising microbubbles having a greater size than the target microbubbles;
removing the second supernatant cake;
applying a third centrifugal field having a third field strength to the second infranatant for a third duration of time, the third field strength being greater than the second field strength, thereby forming a third supernatant cake comprising at least a portion of the target microbubbles and third infranatant comprising microbubbles having a smaller size than the target microbubbles; and
isolating the third supernatant cake.

3. The method of claim 1, wherein the applied first centrifugal field and the first duration of time are sufficient to cause substantially all the microbubbles in the polydisperse microbubbles having a greater size than the target microbubbles to form the first supernatant cake.

4. The method of claim 2, wherein the applied second centrifugal field and the second duration of time are sufficient to cause substantially all the microbubbles in the first infranatant having a greater size than the target microbubbles to form the second supernatant cake.

5. The method of claim 3, wherein the applied second centrifugal field and the second duration of time are sufficient to cause substantially all the target microbubbles to form the second supernatant cake.

6. The method of claim 4, wherein the applied third centrifugal field and the third duration of time are sufficient to cause substantially all the target microbubbles to form the third supernatant cake.

7. The method of claim 1, further comprising:
redispersing the isolated second supernatant cake into a new dispersion;
applying a third centrifugal field having a third field strength to the new dispersion for a third duration of time, thereby forming a third supernatant cake comprising at least a portion of the target microbubbles; and
isolating the third supernatant cake.

8. The method of claim 2, further comprising:
redispersing the isolated third supernatant cake into a new dispersion;
applying a fourth centrifugal field having a fourth field strength to the new dispersion for a fourth duration of time, thereby forming a fourth supernatant cake comprising at least a portion of the target microbubbles; and
isolating the fourth supernatant cake.

9. The method of claim 1, wherein the total volume fraction of the microbubbles in the polydisperse microbubbles suspension and in the first supernatant is equal to or below about 20%,
wherein at least applying one of the first and the second centrifugal fields comprises first determining the centrifugal field strength to be applied using a Stoke's equation, the Stoke's equation correlating the rise velocity of a microbubble in a suspension relative to the bulk fluid under creeping flow conditions with the size of the microbubble, the centrifugal field strength to be applied, and the effective viscosity of the suspension,
wherein the determination of the respective centrifugal field strengths is based at least on the duration of time during which the respective centrifugal field is to be applied.

10. The method of claim 1, wherein the polydisperse microbubbles include microbubbles having sizes from smaller than about 1 μm to larger than about 10 μm.

11. The method of claim 1, wherein the target microbubbles have a size range of about 4 μm to about 5 μm.

12. The method of claim 1, wherein the target microbubbles have a size range of about 1 μm to about 2 μm.

13. The method of claim 1, wherein the polydisperse microbubbles are coated at least in part with lipids.

14. The method of claim 1, wherein the polydisperse microbubbles are coated at least in part with polymeric surfactants.

15. The method of claim 1, wherein the core of the polydisperse microbubbles comprises perfluorobutane.

16. The method of claim 1, wherein the polydisperse microbubbles are obtained by sonication.

17. The method of claim 1, wherein the polydisperse microbubbles have a multimodal size distribution.

18. The method of claim 1, wherein at least one of the polydisperse microbubbles suspension and the first supernatant is placed in a syringe when being subjected to the respective first or the respective second applied centrifugal field, the syringe having a longitudinal axis, a length, a cap, and a drainage portion, wherein the drainage portion is positioned further away from the center of the centrifugal field relative to the cap.

19. Monodisperse microbubbles having a predetermined size range prepared by any one of the methods of claim 1 or claim 2.

20. A method of performing high frequency ultrasonic imaging, comprising:
administering monodisperse microbubbles having an number-averaged size of between about 1 to 10 μm to an animal; and
performing ultrasonic imaging on the animal at a fundamental frequency of at least about 30 MHz.

21. The method of claim 20, wherein the monodisperse microbubbles have a size range of about 1 μm to about 2 μm.

22. The method of claim 20, wherein the monodisperse microbubbles have a size range of about 4 μm to about 5 μm.

23. The method of claim 20, wherein the monodisperse microbubbles have a size range of about 6 μm to about 8 μm.
